(19) United States
(12) Patent Application Publication
(10) Pub. No.: US 2024/0060959 A1
(43) Pub. Date: Feb. 22, 2024

(54) CONTINUOUS METHANE MONITORING DEVICE

(71) Applicant: Arkea Bio Corp., Charlestown, MA (US)

(72) Inventors: Matthew Dunn, Louisville, CO (US); Colin South, Reno, NV (US); Eric Beppler, Broomfield, CO (US); Jaclyn Halula, Broomfield, CO (US); Brian Merrill, Boulder, CO (US); Eric Layton, Broomfield, CO (US); Tanner Leise, Frederick, CO (US)

(21) Appl. No.: 18/235,622

(22) Filed: Aug. 18, 2023

Related U.S. Application Data
(60) Provisional application No. 63/371,949, filed on Aug. 19, 2022.

(51) Int. Cl. G01N 33/497

(52) U.S. Cl. CPC G01N 33/497; G01N 2033/4975

(57) ABSTRACT

A methane monitoring device including a harness component, the harness component configured to adjustably and releasably fix to a ruminant, a monitoring component releasably fixed to the harness component, the monitoring component disposed proximate the ruminant's nostrils and further including a nostril cover disposed proximate the ruminant's nostrils, at least one sensor configured to detect methane concentration in air, a data storage component in electrical communication with the sensor, the data storage component configured to store data.

### CROSS-REFERENCE TO RELATED APPLICATIONS

This application claims the benefit of and priority to U.S. Provisional Patent Application No. 63/371,949, filed on Aug. 19, 2022, the entire contents of which is incorporated by reference herein.

### BACKGROUND OF THE DISCLOSED SUBJECT MATTER

### Field of the Disclosed Subject Matter

The disclosed subject matter relates to a system methane monitoring device. Particularly, the present disclosed subject matter is directed to continuous methane monitoring of exhaled air from ruminants, such as livestock, e.g., cattle.

### Description of Related Art

Ruminants, such as livestock produce methane gas by exhalation and other means during their digestion of plant life. There is a need to accurately monitor, and reduce, said methane gas production for both animal husbandry efficiency and global warming concerns, among others. A vaccine may produce a reduction in said methane production, however there is no cost-effective way to monitor the efficacy of said vaccine in ruminants.

There thus remains a need to monitor the methane production of ruminants in exhaled air over time, without restricting movement of the animal, and receive accurate and time-stamped data which may aid vaccine efficacy/production, as well as the calculation of carbon credits based on a quantifiable reduction in methane gas production. One technique for attaching a monitoring device to an animal is disclosed in U.S. Patent Publication No. 2023/0038208, which is hereby incorporated by reference in its entirety.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a methane monitoring device including a harness component, the harness component configured to adjustably and releasably fix to a ruminant, a monitoring component releasably fixed to the harness component, the monitoring component disposed proximate the ruminant's nostrils and further having a nostril cover disposed proximate the ruminant's nostrils, at least one sensor configured to detect methane concentration in air, a data storage component in electrical communication with the sensor, the data storage component configured to store data.

In some embodiments, the monitoring component may include an oxygen sensor and/or a carbon dioxide sensor.

In some embodiments, the monitoring component may include a pressure sensor.

In some embodiments, monitoring component may include a relative humidity sensor and a temperature sensor.

In some embodiments, the monitoring component may include a transceiver in electrical communication with the data storage component and configured to transmit the data.

In some embodiments, the monitoring component may include an accelerometer.

In some embodiments, the system further comprising a neck board, the neck board releasably coupled to the harness component.

In some embodiments, the neck board may include an ambient air intake and at least one of an oxygen sensor, a carbon dioxide sensor, a pressure sensor, a relative humidity sensor, a temperature sensor, or an accelerometer.

In some embodiments, the neck board may include an electrical power component electrically coupled to a charge management controller.

In some embodiments, wherein the monitoring component may include an intake duct in fluid communication with the nostril cover, the intake duct coupled to a fan, the fan configured to force gas through the intake duct.

In some embodiments, the intake duct is in fluid communication with an airflow sensor.

In some embodiments, the monitoring component may include a filter.

To achieve these and other advantages and in accordance with the purpose of the disclosed subject matter, as embodied and broadly described, the disclosed subject matter includes a methane monitoring device including a ring component having an inner diameter and an outer diameter, defining a radial thickness therebetween, the ring component further having a first ring portion having an arcuate shape and a second ring portion having an arcuate shape, each of the first and the second rings portions having a first and a second ends, wherein the second ring portion is configured to hingedly rotate relative to the first ring portion at the first end, and releaseably couple to the first ring portion at the second end, a housing coupled to an inner perimeter of the first ring portion, the housing having a generally planar first wall and second wall forming a chord of the ring component, defining a cavity disposed therebetween, at least one sensor disposed within the housing, the at least one sensor configured to detect a concentration of methane, wherein the first ring portion has a piercing component.

In some embodiments, the first ring portion has an arcuate hollow portion therein.

In some embodiments, at least one filter is disposed within the arcuate hollow portion.

In some embodiments, the housing may include at least a second sensor disposed therein.

In some embodiments, the housing may include a fan configured to displace a gas within at least a portion of the ring component or the housing component.

In some embodiments, at least one sensor may include an oxygen sensor and a carbon dioxide sensor.

In some embodiments, at least one sensor may include a pressure sensor, a relative humidity sensor or a temperature sensor.

In some embodiments, the monitoring component may include an accelerometer.

To achieve these and other advantages and in accordance with the purpose of the disclosed subject matter, as embodied and broadly described, the disclosed subject matter includes a method for monitoring a gas, the method including installing the gas monitoring on a ruminant; and measuring at least one first parameter of a gas emitted from the ruminant over a first period of time.

In some embodiments, the gas is a greenhouse gas or precursor thereof.

In some embodiments, the gas is methane.

In some embodiments, the ruminant is a cow or a sheep.

In some embodiments, the gas is exhaled or eructed from the ruminant.

In some embodiments, the method further includes administering one or more compositions to the ruminant.

In some embodiments, the one or more compositions comprises at least one of a feed additive, a small molecule inhibitor, or a vaccine.

In some embodiments, the method further includes measuring at least one second parameter of the gas emitted from the ruminant over a second period of time.

In some embodiments, the first period and the second period of time are between approximately one day and a year.

In some embodiments, the first period and the second period of time are between approximately two weeks and six months.

In some embodiments, the at least one first parameter and second parameter are an amount of methane emitted from the ruminant.

In some embodiments, measuring the amount of methane emitted from the ruminant comprises integrating between approximately 10-100% of the measurements over the first period or the second period.

In some embodiments, the method further includes determining a differential amount of greenhouse gas produced in the second period compared to the first period.

In some embodiments, the method further includes determining an amount of mitigated greenhouse gas and/or precursors thereof in response to the administration of the at least one composition to the ruminant between the first period and the second period.

In some embodiments, the method further includes calculating a carbon credit based on the amount of mitigated greenhouse gas and/or precursors thereof.

In some embodiments, measuring at least one first parameter or the at least one second parameter of a gas emitted from the ruminant comprises measuring at a frequency of approximately 0.1-1000 samples per second over the first period or the second period.

To achieve these and other advantages and in accordance with the purpose of the disclosed subject matter, as embodied and broadly described, the disclosed subject matter includes a method for monitoring the health or productivity of a rumen, the method including installing a gas monitoring device on a ruminant having a rumen, measuring at least one parameter of a gas emitted from the ruminant, measuring at least one parameter of the ruminant, calculating a health or productivity of the rumen based on the at least one parameter of the gas, normalizing the health or productivity of the rumen to the at least one parameter of the ruminant and producing a rumen health or productivity value.

In some embodiments, the gas is a greenhouse gas or precursor thereof that has been exhaled or eructed by the ruminant.

In some embodiments, the gas is methane.

In some embodiments, the at least one parameter of the ruminant is a temperature, a standing time, a laying time, a feeding time, a quantity eaten, a drinking time, or a quantity drank.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1A-1C are various views of a methane monitoring device installed on the head of a ruminant in accordance with the disclosed subject matter.

FIGS. 2A-2B are isometric and exploded views of a monitoring component for a methane monitoring device in accordance with the disclosed subject matter.

FIG. 2C is an isometric view of a printed circuit board and components disposed thereon in accordance with the disclosed subject matter.

FIG. 2D is an isometric view of the PCB installed in a housing of the monitoring component in accordance with the disclosed subject matter.

FIGS. 3A-3B are isometric and exploded views of a neck board assembly for a methane monitoring device in accordance with the disclosed subject matter.

FIGS. 3C-3D are isometric and planform views of a printed circuit boards for a neck board assembly in accordance with the disclosed subject matter.

FIGS. 4A-4B are isometric section views of a nose ring, embodiment of a methane monitoring component in accordance with the disclosed subject matter, with the internal circuitry/components of the device shown.

FIG. 4C is an isometric view of an embodiment of a methane monitoring component as shown in FIGS. 4A-B, with a cover attached.

FIG. 4D is an exploded view of an embodiment of a methane monitoring component as shown in FIGS. 4A-C in accordance with the disclosed subject matter.

FIG. 4E depicts various orthogonal and perspective view of an embodiment of a methane monitoring component installed on an exemplary ruminant in accordance with the disclosed subject matter.

FIG. 5A is a 2-D schematic representation of the continuous methane monitoring device in accordance with the disclosed subject matter.

FIG. 5B is a 2-D side view of a schematic representation of the continuous methane monitoring device shown in FIG. 5A.

FIGS. 6A-6B depict exemplary methods for methane monitoring in flow chart form in accordance with the disclosed subject matter.

FIG. 7 depicts a computing node according to various embodiments in accordance with the disclosed subject matter.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

The methods and systems presented herein may be used for monitoring and analyzing animal exhalation. The disclosed subject matter is particularly suited for continuous monitoring of methane included in a ruminant's exhaled air. Ruminants include, but are not limited to, cattle (e.g., large domesticated ruminant animals, e.g., cows (including dairy cattle), bulls), all domesticated and wild bovines (i.e., those belonged to the family Bovidae; e.g., cows, bulls, bison, yaks, African buffalos, water buffalos, antelopes), goats, sheep, giraffes, deer, caribou, and gazelles. As used herein, the term “ruminant” includes ruminant-like animals or pseudo-ruminant animals such as macropods, llamas, camels, and alpacas.

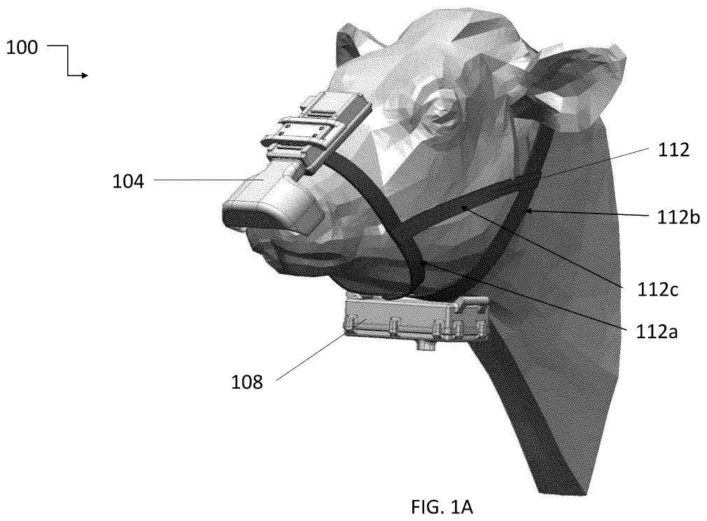

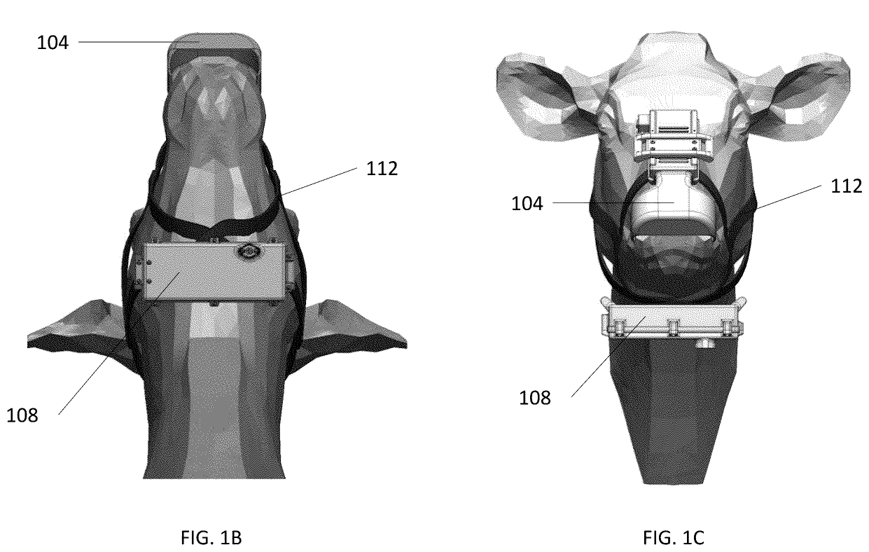

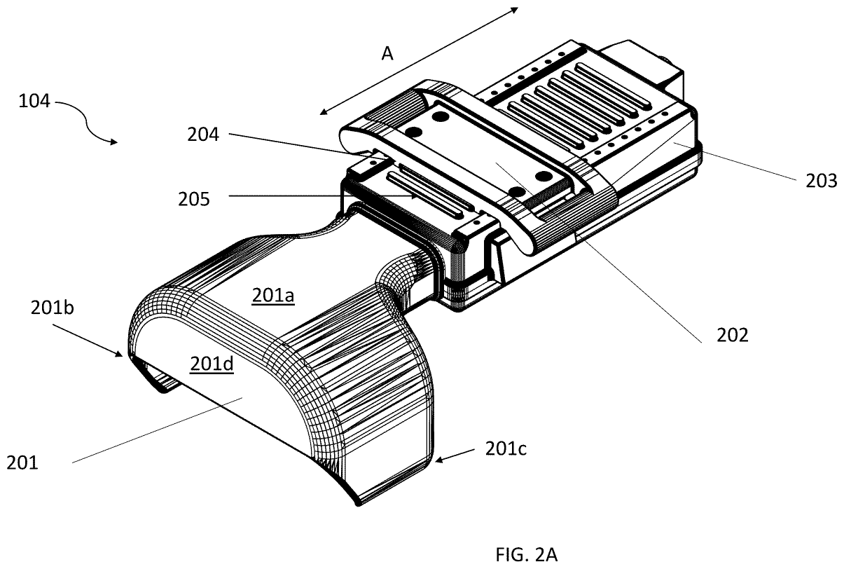

For purpose of explanation and illustration, and not limitation, an exemplary embodiment of the system in accordance with the disclosed subject matter is shown in FIGS. 1A-B and is designated generally by reference character 100. The exemplary animal depicted in these figures is a cow for illustration purposes only, as the apparatus disclosed herein is suitable for use on a variety of animals as noted above. Similar reference numerals (differentiated by the leading numeral) may be provided among the various views and Figures presented herein to denote functionally corresponding, but not necessarily identical structures.

Any device described herein may be utilized on a ruminant within a standard grazing area and under standard grazing conditions. Any device and method described herein may be located in an intensive production environment such as feedlots or grazed productions environments such as ranges. In various embodiments, the devices and methods may be utilized in grazed production environments where low-touch measurement technologies with little large scale infrastructure is needed to monitor the animals, as described herein. In various embodiments, the time period of measurement may be any described herein, such as one day to one year, such as one to six months or more. Any device described herein may be used in a commercial cattle environment or commercial livestock environment. In various embodiments, any device and system described herein may be configured for use in outdoor weather conditions, including but not limited to rain, wind, dust, and frost. In various embodiments, any device or system described herein may be configured for continuous operation of at least 60 days. In various embodiments, continuous operation may include no intervention at the system's nominal sampling and reporting intervals. The systems and devices described herein may be configured for use in −10-50 degrees C., relative humidities between 5-95%, non-condensing and ambient atmospheric pressures of 80-120 kPa. The system described herein may be between 1-10 lbs. The system described herein may be approximately 5 lbs. The system and device may self calibrate for up to 20 minutes each 12-hour period.

Referring now to FIG. 1A, a representation of system 100 releaseably coupled to a ruminant head is shown in isometric view. System 100 includes a monitoring component 104 disposed on the snout of the ruminant. Monitoring component 104 may be coupled to the ruminant's head via a harness 112. System 100 includes a neck board 108 releasably coupled to the head of the ruminant. Neck board 108 may be disposed on the underside of the ruminant's head, proximate the neck area and throat, which can be advantageous in that it minimizes irritation to the animal and permits the animal to raise/retract its head with a full range of motion. In various embodiments, neck board 108 may be disposed proximate another part of a ruminant's body, such as the back of the head, body, leg or the like. Neck board 108 may be coupled to harness 112 such that neck board 108 is open to ambient air and not trapped against the hide or fur of the ruminant. Monitoring component 104 and neck board 108 may both include sensors of the same or different type.

For example and without limitation, monitoring component 104 may measure a methane concentration and neck board 108 may measure a quantity of ambient air. One or both of monitoring component 104 and neck board 108 may be communicatively connected to one another or an off site computing system. For example and without limitation, one or both of monitoring component 104 and neck board 108 may measure and store data related to exhaled gas and ambient air, and transmit said data in the form of electrical signals wired or wirelessly to a computing system. For example and without limitation, one or both of monitoring component 104 and neck board 108 may transmit the data to a data storage component or a computing system based on a geolocation of the ruminant, for example when the ruminant enters a barn or a certain radius from the computing system. Neck board 108 will be described in greater detail herein below. The system may have a Bluetooth low energy link to allow for wireless, automated export of collected data at predetermined periodic intervals. The system may have onboard, non-volatile storage suitable for retaining at least 60 days of continuous sampling at the nominal data rates, such as a micro SD card as will be described herein.

In various embodiments, any sensor or device as described herein may be capable of self-zeroing or zeroing as a response to an interaction with a computing device or a user. For example and without limitation, the system or device may be configured to connect to a control device, docking station or base station. For example and without limitation, the system and devices described herein may be configured to be calibrated, allowing for a known gas to be used to determine one or more coefficients for the gas. Calibration may be automatic or manual. The system may have more than one operating mode, such as 3 operating modes: Calibration, Zeroing, and Normal Operating Mode. Calibration may be used to input calibration gasses, animal information, etc. When in calibration mode the system shall have a method to intake gas concentrations as they are injected into the system. (i.e. input the O2 concentration % as it is used). Zeroing may indicate the device is not on an animal and can be used for baseline measurements, drift measurements, or ambient monitoring. The system may be zeroed with 100% Nitrogen gas. Normal operation is intended for use for data collection on the animal. Upon system power on the system shall be fully operational within 2 minutes.

As shown in FIG. 1A, the continuous methane monitoring device includes a harness component 112. Again, reference to “continuous” indicates that the device has the ability to monitor/record/transmit/analyze over a period of time; but periodic operation of the device at select time intervals is also within the scope of the present disclosure. Harness component 112 can be configured to adjustably and releaseably fix device 100 to an individual ruminant's head. In some embodiments, the animal is a ruminant as described herein, preferably a cow. In some embodiments the animal is a cow. In some embodiments the animal is a horse. In some embodiments the animal is a donkey. In some embodiments the animal is a mule. In some embodiments the animal is a bull. In some embodiments the animal is a deer. In some embodiments the animal is a chicken. In some embodiments the animal is a goat/sheep/ram. In some embodiments the animal is a pig/hog/boar. In some embodiments the animal is a rooster. In some embodiments the animal is an ox. In some embodiments the animal is a buffalo. The harness component can be configured to attach to any suitable ruminant, preferably a ruminant, such as cattle (e.g., large domesticated ruminant animals, e.g., cows (including dairy cattle), bulls), all domesticated and wild bovines (i.e., those belonged to the family Bovidae; e.g., cows, bulls, bison, yaks, African buffalos, water buffalos, antelopes), goats, sheep, giraffes, deer, caribou, and gazelles. In preferred embodiments, the ruminant is a cow, a goat, or a sheep. In more preferred embodiments, the ruminant is a cow. One of ordinary skill in the art would appreciate that the continuous methane monitoring device 100 may be configured for use with a plurality of animals/livestock, and this is a non-exhaustive list.

For example and without limitation, harness 112 may include one or more adjustable straps extending at least partially around a ruminant's head and secured with a buckle, such as a quick attach/release plastic buckle with pinch sides. Harness 112 may include one more padded straps configured to cushion the abrasion around the ruminant's head, especially around corners and features of the skull, face, horns, neck, ears, eyes, beak, crest, or other features of the animal. Harness 112 may be formed from nylon, cotton, ballistic nylon, leather, Kevlar, wool, plastics, rubbers, canvas, a combination thereof, or another material not listed herein. Harness 112 may include an elasticity configured to stretch around the features of the ruminant's head and tighten when in place. Harness 112 may include one or more adjustment features like straps and buckles, double D-rings, pull tabs, holes, slots, hooks and loops, or the like. Harness 112 may include one or more settings, sizes, or shapes configured to secure around the head of an animal, such as a ruminant, or a plurality of successive animals, of the same species or otherwise. Harness 112 may include a plurality of attachment points along its length for additional straps/pads/features configured to better secure around a ruminant's head. Harness 112 may be a commercial or custom halter. Harness 112 may include break-away design features configured to break before injury to the animal occurs.

With further reference to FIG. 1A, harness 112 may include a buckle. Buckle may be a quick-attach/release buckle configured to be detached using a pinching motion along parallel tabs. A buckle may include a male and female end, the male end configured to pass partially through the female end and change shape in a manner that does not allow movement in the opposite direction. For example the male end may include tabs that are deflected inward by the female end up until a certain point, where the tabs are then elastically returned to their position and disposed in slots of holes in the female end, thereby securing the two together. A buckle may include a hook and loop configuration. In some embodiments, the buckle may include Velcro. A buckle may include a belt buckle configuration, wherein one side includes holes at regular intervals and one side includes a pin configured to travel through said hole and hold the first side against a buckle. The buckle may include a pincer configuration wherein a strap is pinched between two metal tongues, thereby securing two sides of the harness together.

In the exemplary embodiment shown in FIG. 1A, the harness 112 includes two loops: a smaller loop 112a (for positioning behind the animal's mouth); and a larger loop 112b (for positioning behind the animals ears); and a cross strap 112c connecting the two loops 112a,b. Thus, the harness is spaced from the animal's mouth (and eyes) so as to minimize irritation and avoid obstructing the animal's grazing, chewing, drinking, breathing, etc. Harness 112 may be a strap that circumvents the side of the ruminant's head and wraps around the back of the horn portion, past the ears. The harness 112 can also include a cross member strap that spans the front of the cow's head between and over the eyes and in front of the horns that anchors the device 100 and/or monitoring component 104 snugly around the pronounced horn portion. Monitoring component 104 is shown extending down the snout of the ruminant and extending over the front of the snout such that a nostril cover (referenced by reference character hereinbelow) is disposed proximate the ruminant's nostrils and mouth.

Thus, in accordance with an aspect of the disclosure, the harness is configured to position the monitoring component 104 in direct fluid communication with the animal's nostril, but spaced a distance (e.g. 0.5˜20 mm) away from the animal to minimizes/avoid direct contact with the nostril. This reduces irritation of the animal, and therefore reduces the likelihood that the animal resists being fitted with the apparatus, and/or seeks to break or dislodge the apparatus.

Harness 112 may be configured to extend around the jaws of the ruminant, herein shown as a cow, but behind the mouth as to not interfere with the cow's eating, breathing, or vocalizations. Additionally, the harness can be elastic to allow for a range of motion to accommodate routine movement (e.g. chewing). These embodiments are for illustration only, and one of ordinary skill in the art would appreciate that any arrangement of straps, pads, helmets, hooks, or other features may be used to secure monitoring component 104 proximate the cow's nostrils as well as other components hereinbelow described.

With continued reference to FIG. 1A, system 100 includes monitoring component 104. Monitoring component 104 may be one or more housings configured to be releaseably fixable to harness 112. Monitoring component 104 may include one or more plastic housings with a plurality of cutouts within them, each cutout corresponding to one or more components as described herein. Monitoring component 104 may be formed from one or more plastics, rubbers, metals, composites, or a combination thereof. Monitoring component 104 may be cast, molded, carved, machined, additively manufactured (3D printing), built, assembled, or otherwise formed.

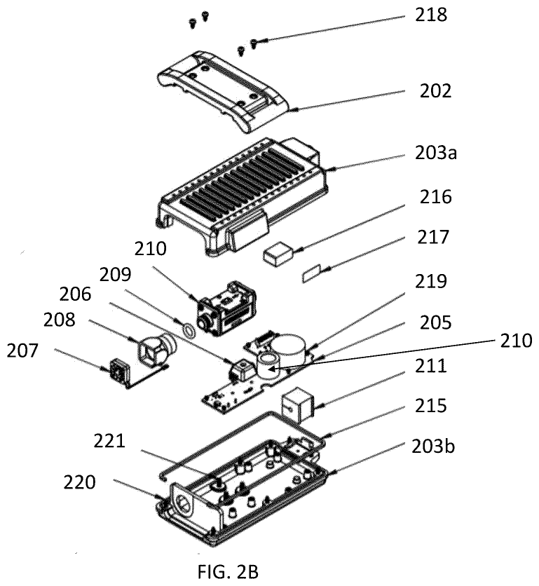

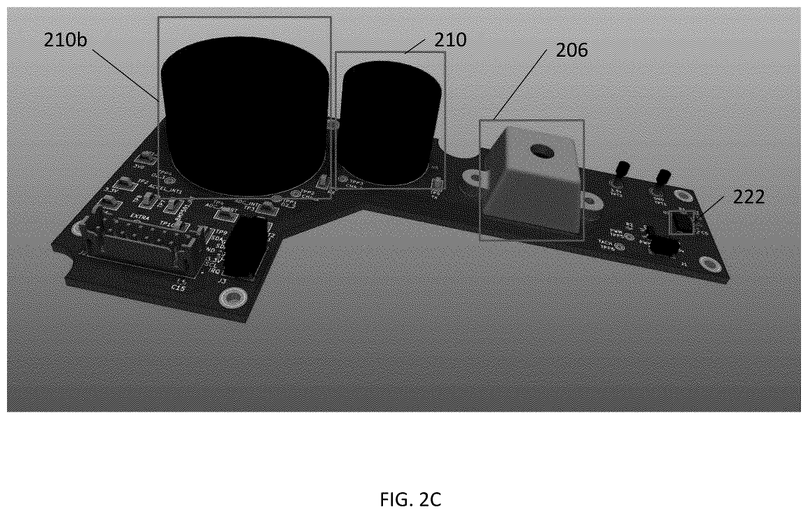

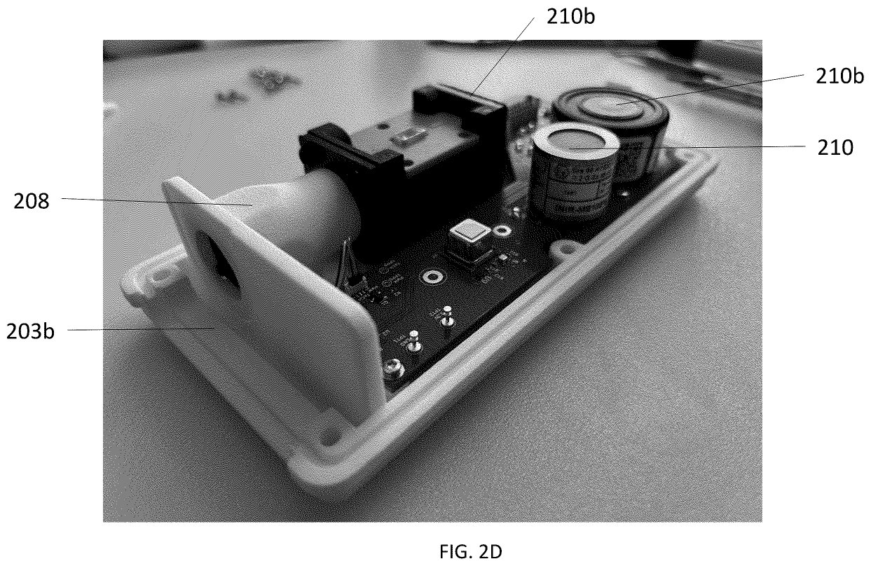

Monitoring component 104 may be coupled to harness 112 via webbing, one or more slots or the like configured to receive the strap, such as a bracket 202, as shown in 2A and 2B. Bracket 202 may be configured to mount adjustably along a housing 203 or portion thereof. For example, housing 203 may have a rail or other geometric feature configured to coupled to the bracket 202 and adjust for the ruminant's head and face. Bracket 202 may be coupled to housing 203 via mechanical fasteners, such as screws and the like. In various embodiments, bracket 202 may be coupled to the housing 203 via a press fit or other geometric feature, such as protrusions and recesses. In the exemplary embodiment shown in FIG. 2A, the bracket 202 has downwardly extending legs 204 that interlock (e.g. press fit; tongue & groove coupling, etc.) with one or more protruding ribs 205 on the upper surface of the housing. This allows for the bracket to be adjusted forward/aft on the animal's face (to thereby adjust position of the monitoring component relative to the nostrils) by sliding the backet along the “A” axis, as shown.

Monitoring component 104 may include a plurality of weight-saving features such as cutouts, carve outs, recesses, or the like. Monitoring component 104 may include one or more ribs assembled into it or manufactured to increase strength. Monitoring component 104 may include a quick attach feature. Quick attach feature may allow a user to quickly detach the “smart” component of the device 100 from harness 112, which may still be attached to the ruminant, and perform a plurality of functions and/or switch the monitoring device 104 with another. Quick attach feature may include a buckle, pull tab, push button, or other elastically deformed component configured to secure with a recess until deflected back and removed. Quick attach feature may be configured for use with one hand, such as a pinching motion. Quick attach feature may be configured to secure into harness 112 with one hand, such as slidably connecting.

Referring now to FIGS. 1B-1C, system 100 is shown a bottom planform view and a frontal view, respectively. One of ordinary skill in the art would appreciate that this is a different view of the device as shown in FIG. 1A, and therefore includes at least all of the components as described with reference to FIG. 1A. This bottom and frontal view shows the device including contours that may be complementary to the slope and shape of the head of a ruminant, herein depicted as a cow, for example. One of ordinary skill in the art would appreciate that the system 100 (including harness 112 and monitoring component 104/neck board 108) may be shaped for use with a certain species, or for an individual animal. FIG. 1B-1C shows harness 112 including a cross member strap configured to cross under the jaw section of the cow's head. In various embodiments, harness 112 may be configured to wrap around the horns or horn section of the ruminant's head, whether ruminant's horns are present or not (such as in cow versus a bull embodiment or other male/female species morphology). The monitoring device 104 and neck board 108 may be installed on harness 112, utilizing a quick attach feature (not shown). According to embodiments described herein above, the photovoltaic cell, if present would be on the top most surface as that corresponds to the topmost face of the system 100, such as on the upper housing of monitoring component 104.

Referring now to FIG. 2A, monitoring component 104 as shown in FIGS. 1A, 1B, may include nostril cover 201. Nostril cover 201 may include one or more cutouts within monitoring component 104. Nostril cover 201 may be one or a plurality of openings configured to intake a gaseous sample, such as air. In some embodiments, nostril cover 201 is configured to be exposed to the air when device 100 is in use, such that it is not sandwiched between the device and the ruminant's skin. Nostril cover 201 may be between 0-15 mm from the surface of the nostrils. In various embodiments, nostril cover 201 may extend forward beyond the proximal end of the animal to be positioned directly above the nostril opening, and extend approximately 5˜10 mm on each side of the nostril. In the exemplary embodiment shown, the nostril cover has a generally planar top wall 201a that extends along the animal's snout and beyond the nostrils, and two arcuate sidewalls 201b,c that extend downwardly on either side of the nostrils. Thus, the nostril cover can have a wider distal end (forming a shroud to ensure coverage of the nostrils) and a more narrow (tapered or stepped) proximal with the sidewalls having chamfered edges to reduce weight and minimize animal agitation. All of the sensors and circuitry of the sensing device can be positioned within the nostril cover shroud.

Nostril cover 201d may also include a generally planar distal wall extending downwardly from the top portion 201a and connecting the sidewalls 201b,c. Thus, the nostril cover 201 serves as an arcuate hollow hood or shield to both position the monitoring component proximate the nostrils, while also blocking ambient air from interfering with (e.g. mixing or diluting) the exhalation from the animal. In various embodiments, nostril cover 201 may extend over the nostrils at a uniform distance, such that the nostril cover mirrors the shapes of the nostril. For example, nostril cover 201 may extend downward over the nostrils as to cover the entire nostril, allowing access to ambient air underneath the hood of the nostril cover 201. In various embodiments, nostril cover 201 may extend downward a greater distance on the sides of each nostril and a lesser distance in front of the nostrils, as to allow for more airflow for the animal. Nostril cover 201 may be expandable or reconfigurable (e.g. walls 201a, b, c can be telescoping or configured with a flexible accordion-style collapsing design) so as to adjust to the size and shape of the ruminant's snout and nostrils. Nostril cover 201 may have various channels formed therein configured to capture gas in the arcuate hood portion and direct said gas into the channels and further into the housing 203.

Nostril cover 201 may be disposed facing the nostrils of the ruminant, such as cow's snout, in some embodiments. Nostril cover 201 may include a separate intake for each of the two nostrils of a ruminant; alternatively, a single intake can receive exhalation from both nostrils. Nostril cover 201 may be configured to mirror the shape of the ruminant's nostrils and/or mouth. In some embodiments, the monitoring component can include a positioning features (e.g., weights distributed at the bottom/center of the device; contoured outer surface to engage animal anatomy) which act to bias/urge the device into a preferred position on the animal such that the nostril cover 201 is properly aligned with the ruminant's orifice(s) (e.g., nostrils).

Nostril cover 201 may include a vapor barrier to prevent humidity in the air from entering the monitoring component 104. Nostril cover 201 may include a humidity sensor configured to measure the moisture in the exhaled air. Nostril cover 201 may include software and/or hardware configured to manipulate/compensate the measurements to account for environmental factors such as humidity when measuring gaseous compounds. For example, and without limitation, nostril cover 201 may include any compensation hardware/software based on the sensor type utilized in said embodiment. Nostril cover 201 may include a plurality of intakes designated for each nostril/mouth. Nostril cover 201 may include a nozzle (e.g., tube or straw) configured to enter the nostrils of the animals to intake exhaled air. Nostril cover 201 may include tubes or straws configured to move in and out of a ruminant's nostrils upon command, upon detection of one or more compounds, or periodically. The nostril cover 201 may include tubes/straws or another form of invasive intakes configured to enter the livestock's nostrils. Nostril cover 201 may extend into monitoring component 104 and include one or more sensors as described herein below.

Nostril cover 201 may include one or more turns configured to manipulate the gaseous sample taken in. For example, the shape of the internal tubing of nostril cover 201 may be used to separate methane from other gases within the exhaled air by gravity or in an active process like an absorber. Housing 203 or nostril cover 201 itself may include one or more fans configured to assist gas in entering the monitoring component 104. The fan used may include a propeller type fan, a bladeless fan, or another type of fluid movement device, like a pump.

With reference to FIG. 2B, housing 203 may include one or more absorption agents configured to capture one or more compounds found within the air taken into the system. The absorption compound may be configured to increase a local concentration of captured compounds/elements for downstream processes. In some embodiments, housing 203 may include a flow meter configured to measure the flow of incoming air into housing 203. The flow meter may be communicatively coupled to one or more computing systems, smartphones, electronic devices, or data storage systems on or off board the device such as neck board 108. The flow meter may be configured to power on or off one or more other components of device 100 based on the measurement of incoming air. For example, and without limitation, flow meter may turn on the intake fans when it detects exhaled breath from the ruminant's nostrils, thereby taking in the air. In various embodiments, all components may be disposed within the center most portion of the internal space of monitoring component 104 and neck board 108 as to protect those components from jostling and damage from day-to-day movement and bumping of the ruminant's head.

In some embodiments, housing 203 may include an outlet. The outlet may be one or more ports within housing 203 configured to exhaust processed air. The outlet may include one or more fans configured to pull air through housing 203. The outlet may include one or more absorption agents configured to capture one or more compounds before exhausting air back into the environment. Outlet may include an open hole, opening, cutout or other pass through in housing 203 such that air may pass through the nostril cover 201 and out of the outlet.

Optionally, the monitoring component 104, neck board 108, harness 112 can include an indicator to confirm (visually, audibly, and/or tactile/haptic feedback) to an operator that the device is properly positioned on the animal with the nostril cover 201 receiving a flow of exhaled methane from the animal. In some embodiments, the device can include an alert/alarm system to notify the operator if/when the device is misplaced, which can be determined based on an absent/interrupted flow at the nostril cover 201.

Referring now to 2B, an exploded view of housing 203 of monitoring component 104 is shown in isometric view. Housing 203 may be formed from an enclosure defined by a top cover 203a and a base tray 203b. Top cover 203a and base tray 203b may be fastened with mechanical fasteners 218. Top cover 203a and base tray 203b may have corresponding geometric features configured to form a seal (e.g. hermetic) therebetween. Housing 203 may include a seal 215 disposed between top cover 203a and base tray 203b. Seal 215 may be a rubber gasket configured to seat within a groove and compressed by one or both of top cover 203a and base tray 203b. Seal 215 may circumscribe the perimeter of housing 203, or a portion thereof, as shown in FIG. 2. For example and without limitation, seal 215 may be an O-ring. In various embodiments, seal 215 may be formed from card stock. In various embodiments, seal 215 may be formed from square stock with a side length of 2 mm.

Monitoring component 104 includes a printed circuit board (PCB) 25 disposed within the housing 203. PCB 25 may be attached within housing 203 via one or more mechanical fasteners, or by chemical adhesives suitable for use with PCBs. PCB 25 may be electrically coupled to at least one sensor 210.

Monitoring component 104 includes at least a sensor 210. Sensor 210 may be disposed within at least a portion of housing 203 and in the path of exhaled gas channeled from nostril cover 201. Sensor 210 may be disposed along the wall of a tube within monitoring component 104. In various embodiments, sensor 210 may be disposed proximate intake duct 208. Intake duct 208 may include fan 207 (as described herein above). In various embodiments, fan 207 may be encased in a square casing, the square casing being 17.5 mm squared. In various embodiments, fan 207 may be integral to intake duct 208. In various embodiments, there may be more than one fan 207 in fluid communication with the intake duct 208. In various embodiments, intake duct 208 may be coupled to sensor 210 and sealed with an O-ring 209 therebetween. In various embodiments, O-ring 209 may be formed from EPDM with an inner diameter of 8 mm and an outer diameter of 13 mm and a width of 2.5 mm. There may be O-rings of a same or similar configuration between any fluid conducting components described herein. Sensor 210 may include a plurality of sensors working in tandem such as a sensor suite to detect methane and quantify the amount of methane present in the sampled gas. Sensor 210 may include an infrared sensor, a photoacoustic sensor, ultrasonic sensor, electrochemical sensor, metal-oxide-semiconductor (MOS) sensor, or a combination thereof, among others. One or more other sensors may be used that operate according to semiconductors, oxidation, catalytic reactions, photoionization, infrared, or a combination thereof. For example, and without limitation, sensor 210 may include an electrochemical gas sensor. Electrochemical gas sensors may measure the concentration of a target gas, such gaseous methane by oxidizing or reduction the target gas at an electrode and measuring the resulting current. Sensor 210 may be a photoionization detector (PID) configured to measure volatile organic compounds and other gases in concentrations such as parts per billion to parts per million. Sensor 210 may produce instantaneous readings, operate continuously, and are commonly used as detectors for gas chromatography or as hand-held portable instruments.

Further, any one of at least one sensor 210 may be a photoacoustic and/or ultrasonic sensor. This type of sensor may detect the acoustic emission created when a pressured gas expands in a low pressure area through a small orifice, such as a gas entering the nostril cover 201. The photoacoustic sensor may detect the presence of methane or another gaseous compound and the concentration thereof, whether itself or in tandem with another sensor or sensor suite.

Further, sensor 210 may be a holographic gas sensor. Holographic gas sensor may use light reflection to detect changes in a polymer film matrix containing a hologram. Since holograms reflect light at certain wavelengths, a change in their composition can generate a colorful reflection indicating the presence of a gas molecule. However, holographic sensors require illumination sources such as white light or lasers, and an observer or CCD detector. A holographic sensor is a device that may include a hologram embedded in a smart material that detects certain molecules or metabolites. This detection is usually a chemical interaction that is transduced as a change in one of the properties of the holographic reflection (as in the Bragg reflector), either refractive index or spacing between the holographic fringes. The specificity of the sensor can be controlled by adding molecules in the polymer film that selectively interacts with the molecules of interest. A holographic sensor aims to integrate the sensor component, the transducer and the display in one device for fast reading of molecular concentrations based in colorful reflections or wavelengths.

In various embodiments, sensor 210 may be an airflow sensor. As described herein above, airflow sensor may be electrically coupled to one or more other electrical components and may operate to direct power to said other components when flow is measured. The system may be capable of measuring or calculating airflow over the sensing region for all gasses. The system may be capable of measuring (or calculating) airflow in the range of 350-500 cc/min (0.35-0.5 L/min) over the gas sensing region. The system may have an airflow sensing accuracy of +1-10%. The system may measure airflow at the same sampling rate as the CH4 sensor (0.016667-1 HZ). The system may have an airflow response time of less than 500 ms.

The holographic sensors can be read from a fair distance because the transducer element is light that has been refracted and reflected by the holographic grating embedded in the sensor. For example, sensor 210 may be configured to be sensitive to methane at about ˜100 ppm (parts per million) and delta (or change over a time period) of +/−5% concentration.

Monitoring component 104 may include at least one sensor 206. Sensor 206 may be configured to measure one or more similar phenomena as sensor 210. In various embodiments sensor 206 may be configured to measure a different quantity or phenomenon than sensors 210. In various embodiments, sensor 206 may be a CO2 sensor. In various embodiments, sensor 206 may be a humidity or relative humidity sensor.

In various embodiments, sensor 206 may be a barometric pressure sensor. The system may monitor ambient atmospheric pressure. The system may be capable of monitoring or calculating the barometric pressure of any gasses under measurement. The system may be capable of measuring ambient barometric pressures between 80 and 120 kPa. The system shall have an ambient barometric pressure accuracy of +/−5 kPa. The system may monitor barometric pressure changes at a rate of once per minute or report changes of greater than 5%.

In various embodiments, sensor 206 may be a temperature sensor.

In various embodiments, the CO2 sensor may be a SCD41-D-R2 sensor. The system may measure emitted CO2 levels from the nostrils of the animal under study. The system may measure CO2 levels between 0 and 5,000 PPM and report the measurement in PPM. The system may have an accuracy of +/−5% or better when measuring CO2. The system may a drift of less than 1% month over month at 2% CO2. The system may monitor CO2 levels at 10 second intervals. The system may monitor CO2 levels at 1 second intervals following the detection of an eating event. The system may respond to changes in CO2 concentrations within 90 seconds. Targeted for functional prototype phase and later. The system may report CO2 with barometric pressure and temperature data such that in post processing compensation can be applied as needed or desired. The system may use atmospheric gas to calibrate the CO2 sensor.

In various embodiments, the barometric pressure sensor may be a BMP581 sensor. In various embodiments, the relative humidity and temperature sensor may be a SHT40-AD1B-R3 sensor. In various embodiments. Further monitoring component 104 may include a cable seal 211. Cable seal 21 may be mechanically or chemically coupled to the housing 203 or the PCB 205. Cable seal 211 may be configured to electrically couple a cable to the PCB 205 or another electrical component described herein. In various embodiments, cable seal 211 may be configured to electrically connect the PCB 205 to one or more computing systems as described herein. In various embodiments, housing 203 may include one or more filters 216 and 217. In various embodiments, one or both of filters 216, 217 may be formed from foam. In various embodiments, said foam may be 60-65 PPI. In various embodiments, one or more of filters 216, 217 may be formed from nylon mesh.

Referring now to FIG. 2C, PCB 205 is shown in perspective view. In various embodiments, PCB 205 includes sensor 210 as described above. As described hereinabove, sensor 210 may be any sensor type. In various embodiments, sensor 210 may be a methane sensor. For example and without limitation, sensor 210 may be a INIR-ME-100%. Methane sensor may be configured to measure or calculate grams per day of methane emitted from an animal on which the device is installed. Methane sensor may be configured to measure methane emitted from the nostrils and/or the mouth of the ruminant. In various embodiments, the methane sensor may be configured to measure methane between 0-4000 PPM and report said level in PPM with an accuracy of +/−5%. The system's response to methane shall not differ by more than 0.2% month over month at 2,000 PPM. The system shall measure methane levels at 1 second intervals continuously. The system shall monitor methane levels at the maximum sampling rate possible following detection of an eating event. The system shall respond to changes in methane levels in 10 seconds or less. The system may use 5% CH4 in Nitrogen to calibrate the high range of the nitrogen sensor. The system may use 2% CH4 in nitrogen to calibrate the mid range of the sensor.

In various embodiments, PCB 205 may be electrically coupled to more than one sensor of the same of varying type. In various embodiments, PCB 205 may be electrically coupled to accelerometer 222. For example and without limitation, accelerometer 222 may be an ADXL343 sensor. The system may be capable of monitoring head acceleration (referred to as movement) for the animal under study. The system may be capable of monitoring jaw movement for the animal under study. The system may monitor movement on 3 orthogonal axes. Targeted for functional prototype phase and later. The system may monitor movement in units of G's. The system may monitor movement between 0 and 5G. The system may monitor movement at a rate of 10 hz. The system may have an accuracy of 0.2Gs on each axis. The system may have a response time for changes to head movement of less than 100 ms. The system may analyze movement data in real time to determine when feeding events (or other relevant activities) are detected. When relevant determinations are made these should be recorded in the data and the relevant sampling rates should be modified until the activity expires.

PCB 205 may be electrically coupled to sensors 206. Sensors 206 may include a cover mechanically coupled to PCB 205 with at least one opening disposed therethrough. As described above, sensors 206 may be a sensor suite disposed within the cover. For example and without limitation, sensors 206 may include a CO2 sensor, barometric pressure sensor, relative humidity and/or temperature sensor. These sensors may be of the make described herein. In various embodiments, these or other sensors may be disposed on PCB 205 without the cover, as shown. In various embodiments, a portion of sensors may be disposed within the cover, and another portion may be disposed exterior to the cover.

In various embodiments, PCB 205 may be electrically coupled to oxygen (O2) sensor 210b. For example and without limitation, oxygen sensor 210b may be an SGX-7OX sensor. The system may monitor emitted O2 levels from the nostrils of the animal under study. The system may monitor Oxygen (O2) levels between 0 and 250,000 PPM and report the measured result in PPM. The system may monitor oxygen levels with an accuracy of +/−5% or better. The system may have a drift of less than 0.2% month over month at 21% O2. The system shall monitor O2 levels at 10 second intervals. The system may monitor O2 levels at 1 second intervals following the detection of an eating event. The system may respond to changes in O2 concentrations within 15 seconds. The system may use 22% oxygen (+/−2% as measured by a commercial O2 concentration gauge) to calibrate the midpoint.

Referring now to FIG. 2D, a perspective view of PCB 205 within base tray 203b is shown. PCB 205 may be electrically coupled to sensors 210, 210b. In various embodiments, airflow sensor 210 may be disposed proximate the PCB 205 and electrically coupled thereto within housing 203. Airflow sensor 210 may be in fluid communication with air intake 208. Sensors 210, 210b are shown as either in line with intake duct 208 or proximate thereto, such as to be exposed to a gas entering the housing 203. In various embodiments, these sensors may be all disposed in direct line with the gas flow, or another configuration. One of skill in the art would appreciate the various configurations the housing and sensors may be suited for system 100. In some embodiments, intake duct 208 may include a straight tube, as shown in FIG. 2D, wherein air is captured and travels through intake duct 208 and contacting sensors 210, 210b, such as an infrared of photoacoustic sensor, then out of an exhaust. This is merely an example of the internal geometry of intake duct 208 and the traveled path of exhaled gas by the livestock and analyzed by the system 100.

With continued reference to FIG. 2D, varying internal tubing is shown in schematic view representing the path gases travels within monitoring component 104. For example, and without limitation, the internal tubing may be substantially straight wherein the gases travel in one direction through the device. In various embodiments, the path may be split by intake duct 208 or within intake duct 208 and configured to meet at a common junction and travel towards one or more sensors or within one or more sensors. In various embodiments

In various embodiments, intake duct 208 may make a plurality of turns, such that exhaled air is taken in from a split intake, and then travels around a turn with a length L and diameter D, then traveling around a second turn and out of an outlet. For the purposes of this disclosure, these turns may be left/right, as in a planform arrangement, or up/down such that the air is traveling against or with gravity.

PCB 305 may include any electrical power component configured to provide electrical energy to the other components of device to power said components. Electrical power component may be a battery such as a replaceable electrochemical battery cell (i.e., D, C, AA or AAA batteries). Electrical power component may include a rechargeable battery such as a lithium ion battery. Electrical power component may include a plug port configured to charge said battery by a wall outlet at the amperage and voltage in a residential home. Electrical power component may include a motion-charged battery configured to charge itself based on the motion of said electrical power component, such as along with the movement of a ruminant's head during daily activity (i.e., eating, drinking, walking, turning of the head, flicking of the ears). Monitoring component 104 and/or neck board 108 may include shielding around electronics, sensors, or other sensitive components, the shielding configured to protect said components from electromagnetic interference such as signals from a transceiver, antenna, or emitter. Electrical power component may be configured to last ˜30 days between charges or battery replacement, in some embodiments. The system shall have adequate onboard power to sustain nominal data collection rates for the usable period, for example 10 amp hours at 12 volts.

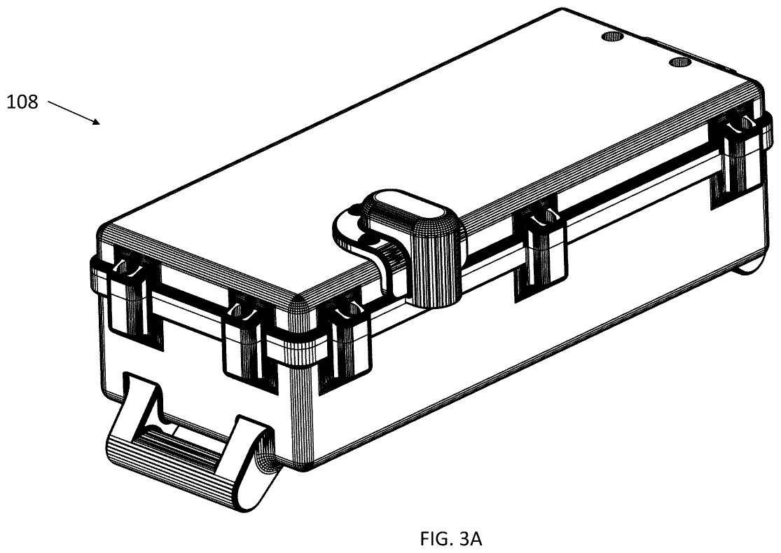

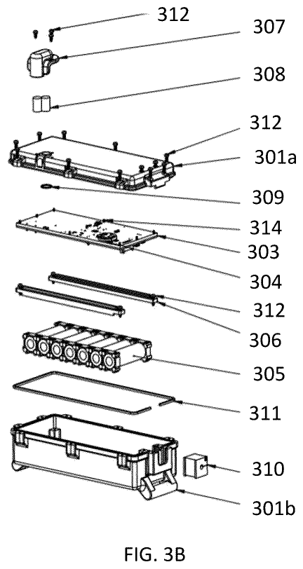

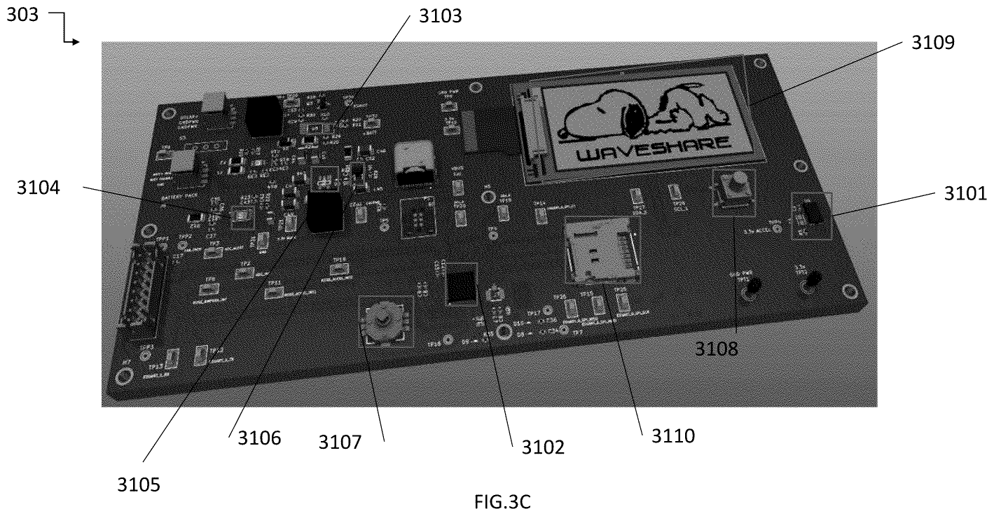

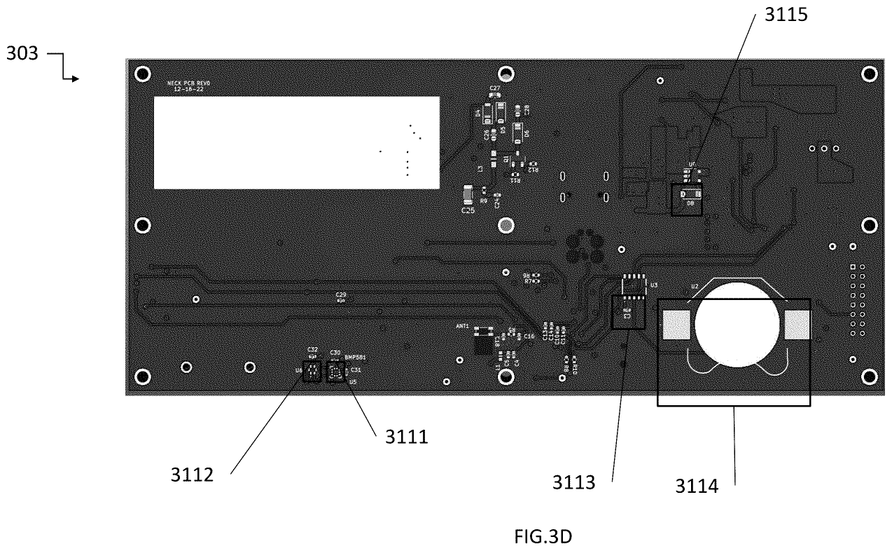

Referring now to FIG. 3A, a perspective view of a neck board 108 is presented. Neck board 108 may be the same or similar to neck board 108 as shown in FIGS. 1A-1B. Neck board 108 may be mechanically coupled to harness 112 in any manner as described herein. Neck board 108 may be enclosed within housing 301, formed from top cover 301a and base tray 301b. For example and without limitation, neck board 108 may be configured to receive a strap of harness 112 through one or more brackets integral to the enclosure, such as integral to base tray 301b. In various embodiments, two brackets may be disposed on a first and second end of base tray 301b. In various embodiments, brackets may be coupled to housing 301. Any component as described herein may be mechanically coupled to housing 301 or PCB 303 via screws 312.

With continued reference to FIG. 3, neck board 108 may include PCB 303. PCB 303 may be mechanically or chemically adhered with housing 301. In various embodiments PCB 303 may be mechanically coupled to housing 301 via one or more mechanical fasteners, such as screws, nails, pegs or the like. In various embodiments PCB 303 may include geometric features that matingly coupled with features in housing 301.

With continued reference to FIG. 3B, neck board 108 may include batteries 304, 305. Batteries 304, 305 may be coin batteries or disposable batteries. In various embodiments, batteries 304, 305 may be rechargeable batteries such as lithium ion batteries. In various embodiments, batteries 304, 305 may be changeable after use, for example, removed from electrical communication with neck board 108 and switched for new batteries. In various embodiments, neck board 108 may include battery straps 306 configured to secure batteries 304, 305 within housing 301. In various embodiments, battery strap 306 may be configured to circumscribe said battery and mechanically coupled to a portion of housing 301.

Neck board 108 may include any electrical power component configured to provide electrical energy to the other components of device to power said components. Electrical power component may be a battery such as a replaceable electrochemical battery cell (i.e., D, C, AA or AAA batteries). Electrical power component may include a rechargeable battery such as a lithium ion battery. Electrical power component may include a plug port configured to charge said battery by a wall outlet at the amperage and voltage in a residential home. Electrical power component may include a motion-charged battery configured to charge itself based on the motion of said electrical power component, such as along with the movement of a ruminant's head during daily activity (i.e., eating, drinking, walking, turning of the head, flicking of the ears). Monitoring component 104 and/or neck board 108 may include shielding around electronics, sensors, or other sensitive components, the shielding configured to protect said components from electromagnetic interference such as signals from a transceiver, antenna, or emitter. Electrical power component may be configured to last ˜30 days between charges or battery replacement, in some embodiments.

With continued reference to FIG. 3, neck board 108 may include ambient air vent 307. Ambient air vent 307 may be coupled to top cover 301a and configured to transmit air from ambient into housing 301. In various embodiments, there may be more than one opening through housing 301 configured to transmit air from ambient into housing 301. In various embodiments, ambient air vent 307 may be mechanically coupled to housing 301. In various embodiments, ambient air vent 307 may be configured to wrap from a top surface of housing 301 to a side surface of housing 301. One of skill in the art would appreciate that when mounted, ambient air vent 307 may be configured to wrap from a bottom surface of the housing 301 to the side of housing 301, as neck board 108 may be mounted upside down relative to its orientation in FIGS. 3A-3B. in various embodiments, ambient air vent 307 may include an ambient air filter 308. For example and without limitation, ambient air filter 308 may be formed from 60-65 PPI foam. In various embodiments, any filter as disclosed herein may be utilized as ambient air filter. In various embodiments, ambient air filter 308 may be configured to remove particulate from the ambient air as it travels through ambient air vent 307 into housing 301. Neck board 108 may include a seal 309. Seal 309 may be any seal as described herein.

For example and without limitation, seal 309 may be an O-ring with a square profile with a 1 mm side length. Neck board 108 may include a cable seal 310. Cable seal 310 may be any seal or cable seal as described herein. For example and without limitation, cable seal 310 may be a grommet formed with a circular opening and a rectilinear outer perimeter. In various embodiments, cable seal 310 may be formed to fit any opening in housing 301 where a cable is run. In various embodiments, cable seal 310 may form a port or a selectively sealable port. Neck board 108 may include a seal 311 configured to form a seal in housing 301 between top cover 301a and 301b. In various embodiments, seal 311 may be any seal as described herein. In various embodiments, seal 311 may be formed as an O-ring. For example and without limitation, seal 311 may be formed as a card stock O-ring. For example and without limitation, seal 311 may be formed with a square cross section with a 2 mm side length.

Referring now to FIG. 3C, a planform view of PCB 303 is depicted. PCB 303 may be any PCB as described herein. One of skill in the art would appreciate this is one example of a layout and assembly of PCB suitable for the system described herein. PCB 303 may include PCB 303 may include and be electrically coupled to accelerometer 3101. Accelerometer 3101 may be configured to determine a relative orientation of PCB 303 in space. For example and without limitation, accelerometer 3101 may be an ADXL343 sensor. In various embodiments, accelerometer 3101 may be any accelerometer described herein. In various embodiments, accelerometer 3101 may be electrically coupled to any component described herein through a wired or wireless communication protocol. PCB 303 may include and be electrically coupled to multiprotocol wireless system on a chip (SoC) 3102. Multiprotocol wireless SoC 3102 may be configured to receive and send any electrical signals between one or more components described herein or offboard, for example over a wireless communication. In various embodiments, multiprotocol wireless SoC 3102 may be configured to perform one or more mathematical or logical operations on any electrical signals present within the system described herein. For example and without limitation, multiprotocol wireless SoC 3102 may be a EFR32MG24B220F1536IM48-B SoC.

With continued reference to FIG. 3C, PCB 303 may include a solar battery charger 3103. In various embodiments, solar battery charger 3103 may be a photovoltaic cell. Further, electrical power component may include a photovoltaic cell configured to charge the battery. A solar cell, or photovoltaic cell, is an electronic device that converts the energy of light directly into electricity by the photovoltaic effect, which is a physical and chemical phenomenon. It is a form of photoelectric cell, defined as a device whose electrical characteristics, such as current, voltage, or resistance, vary when exposed to light. Individual solar cell devices are often the electrical building blocks of photovoltaic modules, known colloquially as solar panels. They can be used as a photodetector (for example infrared detectors), detecting light or other electromagnetic radiation near the visible range, or measuring light intensity.

The photovoltaic cell may be disposed on the top most surface of PCB 303, the top surface facing the sun when in installed on the livestock animal. The photovoltaic cell may be disposed on the monitoring component, on a different part of the livestock (such as on the top of the head, the back, or another area disposed on the animal). The photovoltaic cell, if located off board the monitoring component may be connected via one or more wires to monitoring component. For example and without limitation, solar battery charger 3103 may be a SPV1040TTR solar battery charger.

With further reference to FIG. 3C, PCB 303 may include a barometric pressure sensor 3104. In various embodiments, barometric pressure sensor 3104 may be any pressure sensor as described herein. For example and without limitation, barometric pressure sensor 3104 may be a BMP581 sensor. PCB 303 may include a direct to direct (DC-DC) current step down converter 3105. In various embodiments, DC-DC step down converter 3105 may be a LM3671MF-3.3_NOPB converter. PCB 303 may include a charge management controller 3106. In various embodiments, charge management controller 3106 may be configured to control the electric charge discharged or charged of an electrical component such as an energy storage component, like a battery. For example and without limitation, charge management controller 3106 may be a MCP73831 controller. In various embodiments, PCB 303 may be electrically coupled to a directional joystick 3107. For example and without limitation, joystick 3107 may be a SKRHAAE010.

In various embodiments, PCB 303 may be electrically coupled to and include a push button 3108. Push button 3108 may be configured to power the system, cycle the system or perform another function as a result of a manual interaction with a user. The system may have a method, such as push button 3108, to accept user input to allow for selection of operating mode (calibration, zeroing, normal usage). The system shall have a method of user feedback to allow for selection of operating mode (calibration, zeroing, normal usage).

In various embodiments, push button 3108 may be accessible while in the housing 301, for example through an opening in housing 301. For example and without limitation, push button 3108 may be a KSC1101J_LFS. In various embodiments, one or more electrical signals, measured elements of data or other electrical information may be transmitted to another electrical component on the board or off the board in response to the button depression by a user. PCB 303 may include a screen 3109. Screen 3109 may be an E-ink screen. For example and without limitation, screen 3109 may be MIKROE-3158/MIKROE-EINK model. In various embodiments, screen 3109 may display measurement results from any sensor described herein and electrically coupled thereto. In various embodiments, screen 3109 may be configured to display one or more WIFI connections or alerts for a user. In various embodiments, screen 3109 may be configured to display one or more messages to the user, such as chemical concentration levels or low battery signals. PCB 303 may include a micro SD card adapter 3110. In various embodiments, micro SD card adapter 3110 may be configured to receive and electrically connect the PCB to a micro SD card. In various embodiments, one or more electrical signals that represent elements of data may be transmitted and stored on the micro SD card. In various embodiments, a user may remove the micro SD card from the micro SD card adapter 3110 and replace with a new card. In various embodiments, one or more transceivers may be configured to transmit data stored on the micro SD card to one or more wireless connected components.

In various embodiments, PCB 303 or neck board 108 may include a data storage component. Data storage component may include one or more components located on-board neck board. Data storage component may include one or more chips, processors, solid state drives (SSD), hard disk drives (HDD), flash memory devices, optical storage devices, or a combination thereof configured to store data. The data stored may include one or more of identified gases and/or concentrations thereof. The data may also include environmental information and time stamps for downstream data processing, such as humidity compensation. Data may be digital data that may be machine-readable on a storage medium, such as data storage component. Data may be stored on or in data storage component in raw form, processed, pre-processed, or time stamped such that one or more downstream components or users can visualize the data over time. For example and without limitation, data stored on data storage component may be stored as visual data such as one or more charts. The charts may plot gas detection over time, concentration over time, or some other characteristic of the measured gas or gases and displayed on screen 3109. Neck board 108 may include a port for insertion of a USB, lightning, thunderbolt, or other electronic storage device external to the device. Data storage component may be centrally located off board and configured to receive data from one or more monitoring components working in tandem or alone.

In various embodiments, monitoring component 104 and/or neck board 108 may include a transceiver. Transceiver is configured to transmit data, whether stored data (in data storage component) or instantaneously measured data for immediate transmission. In radio communication, a transceiver is an electronic device which is a combination of a radio transmitter and a receiver, hence the name. It can both transmit and receive radio waves using an antenna, for communication purposes. The term is also used for other devices which can both transmit and receive through a communications channel, such as optical transceivers which transmit and receive light in optical fiber systems, and bus transceivers which transmit and receive digital data in computer data buses. Transceiver may be configured to transmit the data, whether stored or instantaneously after measurement, to one or more off-board storage component such as a centralized database for analysis. Transceiver may be configured to transmit data over a plurality of ranges, such as long range communication over satellites, radio or cell towers, over the internet (such as over a WiFi or Ethernet connection), a combination thereof, or the like.

Referring now to FIG. 3D, a planform view of a PCB 303 is depicted. PCB 303 may include a barometric pressure sensor 3111. Barometric pressure sensor 3111 may be the same or similar to any pressure sensor as described herein. In various embodiments, barometric pressure sensor 3111 may be a BMP581 sensor. PCB 303 may include a relative humidity and temperature sensor 3112. In various embodiments, the relative humidity and temperature sensor 3112 may be any humidity or temperature sensors as described herein. In various embodiments, sensor 3112 may be standalone humidity and temperature sensors. For example and without limitation, relative humidity and temperature sensor 3112 may be a SHT40-AD1B-R3. PCB 303 may be electrically coupled to a real time clock 3113. Real time clock 3113 may be a circuit configured to keep time, such that all measured data from any other sensor as described herein is time stamped. In various embodiments, real time clock 3113 may be configured to record a time of day, day of the week, day of a month, day or month of a year, or a longer timescale. In various embodiments, real time clock 3113 may be configured to communicate a time datum to one or more other sensors communicatively connected thereto. For example and without limitation, real time clock 3113 may be a MAX31329ELB+. The system may include a RTC clock suitable for data timestamping at 10 Hz intervals. The system may be capable of syncing times with a base station to associate timestamped ambient measurements and external interventions with collected data.

PCB 303 may include a battery holder 3114. In various embodiments, battery holder 3114 may be a coin battery holder. In various embodiments, battery holder 3114 may be a disposable battery holder and configured to secure a cylindrical battery. In various embodiments, PCB 303 may include a low voltage drop diode 3115. Low voltage drop diode 3115 may be in electrical communication with any component as described herein. For example, and without limitation, low voltage drop diode 3115 may be a MAX40200AUK.

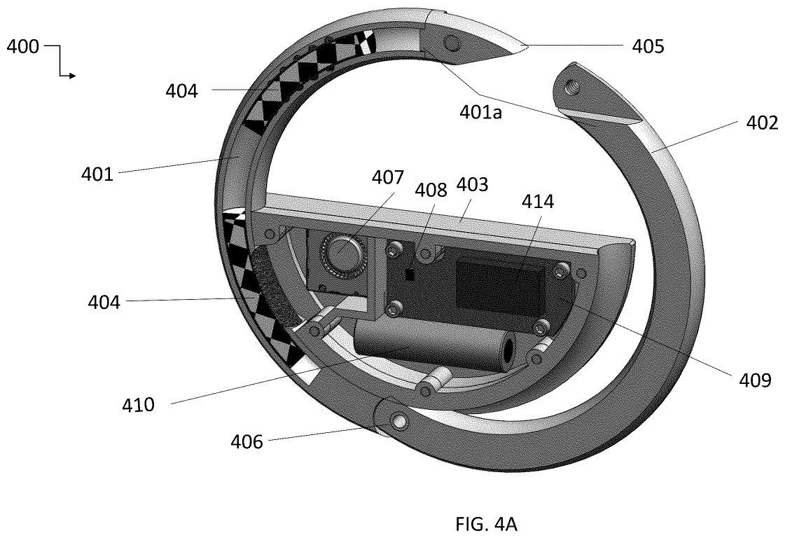

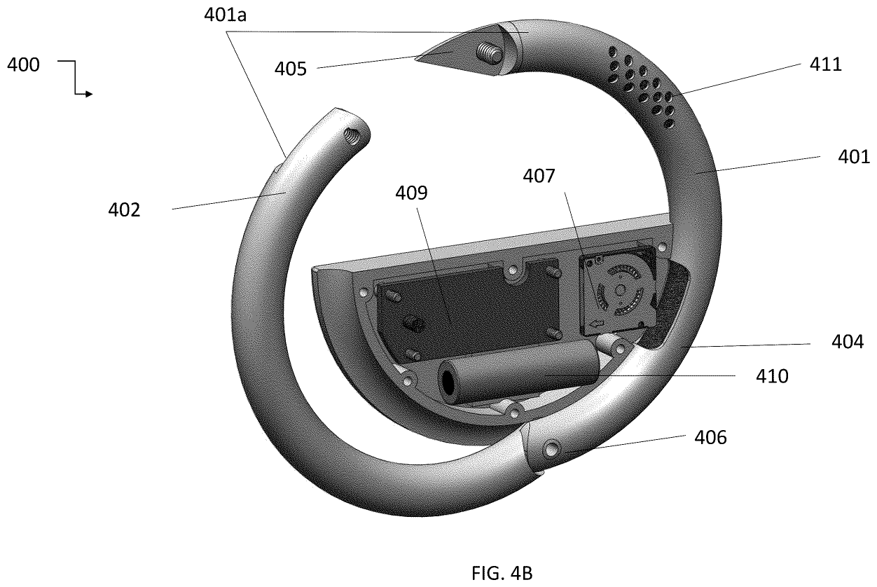

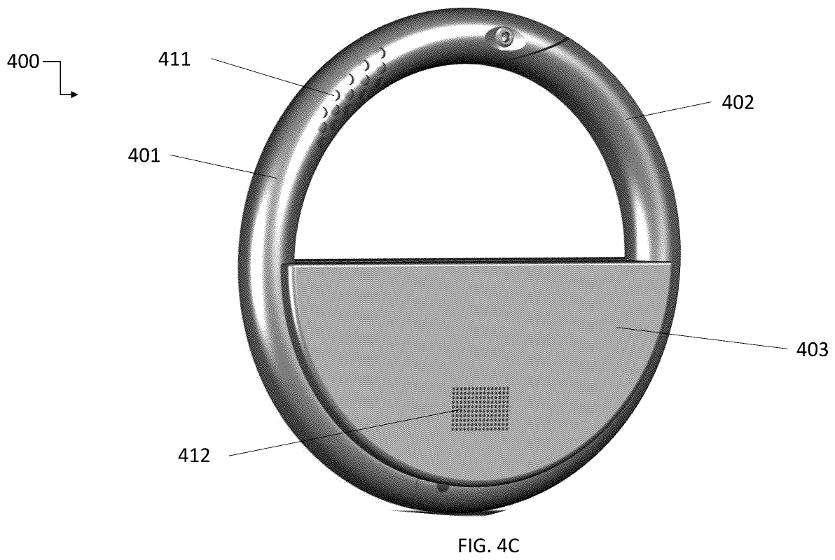

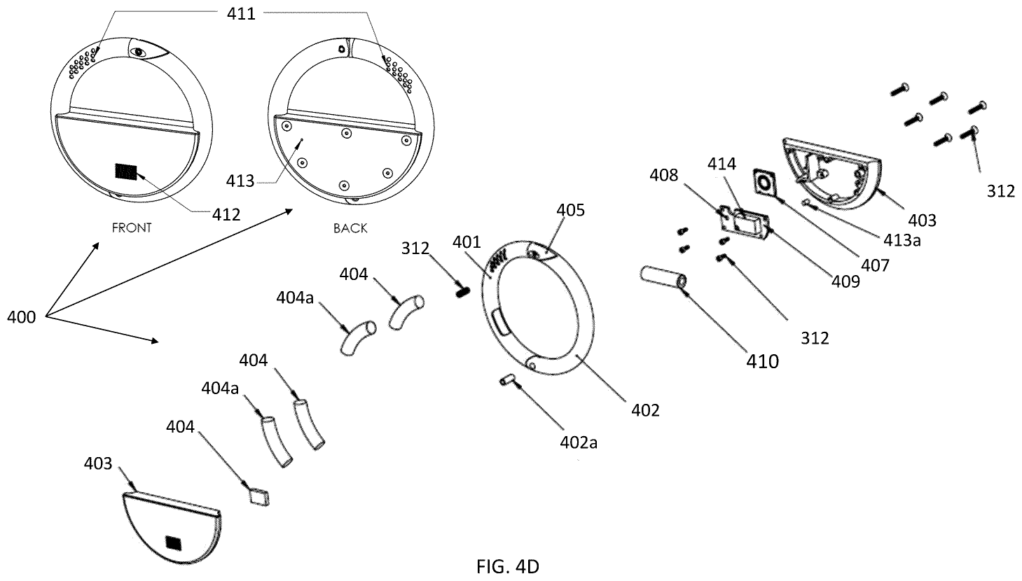

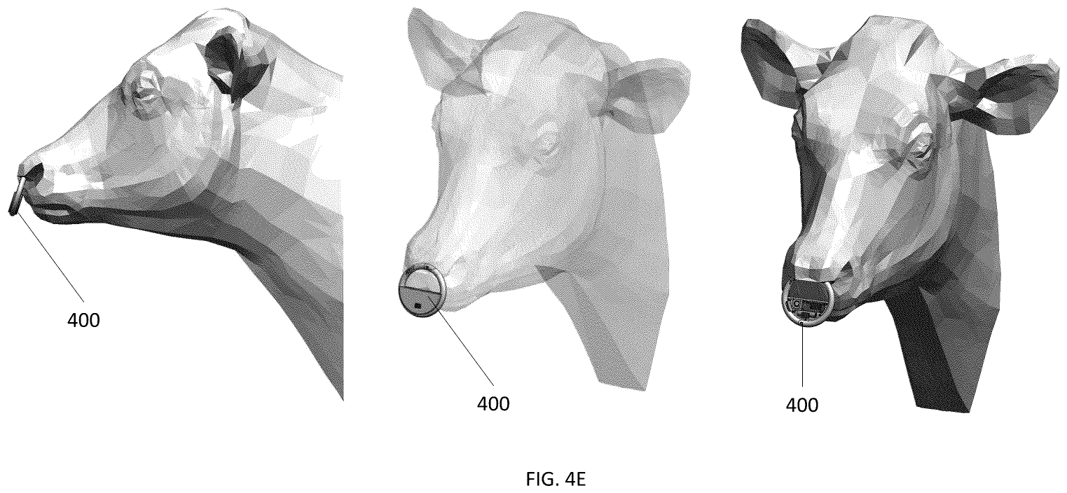

Referring now to FIGS. 4A-4E, a methane monitoring device 400 is shown in a plurality of perspective views, exploded views, alone and installed on an exemplary ruminant. Device 400 may be formed as a ring configured to be placed in a ruminant's snout, such as through the septum of a ruminant's nose. In various embodiments, ring device 400 may have a ring component 401a. Ring component 401a may be circular in planform. In various embodiments, ring component 401a may have an oblong, or elliptical planform shape. In various embodiments, ring component 401a may have a circular cross section. In various embodiments, ring component 401a may have an oblong cross section. In various embodiments, ring component 401a may have a polygonal cross section, forming flat sides on one or more arcuate surfaces of the ring component. In various embodiments, ring component 401a may have a discontinuous arcuate external surface. In various embodiments, ring component 401a may have a continuous and/or smooth arcuate external surface. In various embodiments, ring component 401a may have a constant cross section along its radial distance. In various embodiments, ring component 401a may have a first cross section shape at a first portion of the ring, and a second/distinct cross sectional shape at a second portion of the ring. For example and without limitation, ring component 401a may have a circular cross sectional shape proximate the ruminant's septum, so as to minimize discomfort for the animal and a polygonal cross sectional shape disposed outside of the ruminant's biology.

With continued reference to FIGS. 4A-4E, ring component 401a may have an inner diameter and an outer diameter, defining a radial thickness therebetween. In various embodiments, ring component 401a may have an axial width defined by circular planform walls parallel to the axis extending through the center of the ring equidistant to the ring. Ring component 401a may have a first ring portion 401 having an arcuate shape (e.g. semi-circular) and a second ring portion 402 having an arcuate shape (e.g. semi-circular). In various embodiments, the first and second ring portions may extend along the same radial arc, each forming half of the ring component 401a. In various embodiments, first ring portion 401 may extend a lesser arcuate distance than second ring portion 402.

In various embodiments, first ring portion 401 and second ring portion 402 may be hingedly coupled via a pin 402a or similar mechanical fastener at a first end 406. In various embodiments, first ring 401 and second ring portion 402 may have corresponding geometrical features configured to allow for hinged rotation and mechanically coupling. In various embodiments, first ring portion 401 and second ring portion 402 are releaseably coupled at a second end 405. Second end 405 may include a piercing tip disposed on one or both of first ring portion 401 or second ring portion 402. First ring portion 401 may have a piercing tip configured to pierce the septum of the ruminant. First ring portion 401 may have a mirror image piercing tip as second ring portion 402 to correspondingly mate. In various embodiments, first ring portion 401 and second ring portion 402 may be releaseably coupled via one or more mechanical fasteners such as screws, set screws, pins or the like. In various embodiments, first ring portion 401 may include a hook or other geometrical feature configured to mate with second ring portion 402 at the second end 405 such that the ring component 401a cannot open accidentally.

In various embodiments, ring component 401a may be configured to separate parallel to the axial direction before rotating in the plane parallel to the ring. In various embodiments, ring component 401 may include a hollow cavity disposed therein. In various embodiments, the hollow cavity may be rectilinear cavity disposed in the arcuate first ring portion 401. In various embodiments, the hollow cavity may extend the entirety of first ring portion 401, or a portion thereof. The hollow cavity may include at least one filter 404. Filter 404 may be any filter as described herein. In various embodiments, filter 404 may be configured to filter ambient air through the filter on to one or more components to be described herein. In various embodiments, filter 404 may be configured to filter particulate or moisture out of exhaled gas by the ruminant. In various embodiments, filter 404 may be formed from foam, with a sleeve filter 404a circumferentially enclosing it. Sleeve filter 404a may be hydrophobic. Sleeve filter 404a may be disposed around each filter 404, in various embodiments.

Methane measurement device 400 includes a housing 403 coupled to at least one of first ring portion 401 or second ring portion 402. Housing 403 may be integral to one or both of first ring portion 401 or second ring portion 402. In various embodiments housing 403 may be releaseably coupled to ring component 401a. In various embodiments, housing 403 may be adjustably coupled to ring component 402. In various embodiments, housing 403 may be permanently coupled by welding or chemical adhesion. In various embodiments, housing 403 may be coupled to the inner perimeter of the ring component 401a. In various embodiments, housing 403 may be coupled to an outer perimeter of ring component 401a and extend through the inner portion of the ring.

Housing 403 may include a groove configured to receive the second ring portion 402 when it hingedly rotates inward to couple with first ring portion 401. In various embodiments, housing 403 may be formed by two parallel walls extending from first ring portion 401 to second ring portion 402 with a complementary arcuate section extending along the arcuate portions of the rings, and a linear wall forming a chord (or diameter extending between the two ring components, when a circular embodiment is employed) of the ring component 401a. In various embodiments, housing 403 may be half of the interior volume of the ring component 401a. In various embodiments housing 403 may be less than half the interior volume of the ring component 401a. In various embodiments, housing 403 may be wholly disposed within the ring component 401a, thereby unintrusive to the inner volume of the ring and out of the ruminant's way. In various embodiments, housing 403 may be more than half of the volume of the interior of the ring component 401a, leaving suitable room for the piercing tips to pierce the septum of the ruminant. Housing 403 may have hollow cavity disposed therein. Housing 403 may include a hollow cavity mirroring the outer mold line of housing 403. In various embodiments, housing 403 may have a rectilinear hollow cavity disposed therein. In various embodiments, housing 403 may have a circular or oblong hollow cavity disposed therein.

With continued reference to FIGS. 4A-4E, housing 403 may have a fan 407 within the hollow cavity. Fan 407 may be any fan as described herein, such as a square fan with a 17.5 mm side. In various embodiments, there may be more than one fan 407 disposed proximate the first ring portion 401 channel configured to pull gas through said channel and into the housing 403. Fan 407 may be vented to the ambient air through at least an opening in the housing 403. In various embodiments, fan 407 may be mounted to the wall of the housing 407 and forming a part thereof. For example, the fan may be emplaced within an opening in the wall, forming a portion of the enclosure with its own casing. In various embodiments, fan 407 may be mounted to an interior wall of the housing 407. In various embodiments, fan 407 may be mounted within the channel within first ring portion 401.

With continued reference to FIGS. 4A-4E, housing 403 may have a hollow cavity disposed therein, inside the hollow cavity at least one sensor 408 disposed therein. Sensor 408 may be configured to detect a concentration of methane in an exhaled gas or ambient air. In various embodiments, sensor 408 may be configured to detect any or measure any gas as described herein, namely oxygen, hydrogen, carbon dioxide, methane or other gases. In various embodiments, sensor 408 may be an airflow sensor, methane sensor, oxygen sensor, carbon dioxide sensor, hydrogen sensor, temperature sensor, humidity sensor or movement sensor.

In various embodiment, airflow sensor may be configured to ensure each sensor receives mixed air at the appropriate flow rate and temperature for operation. In various embodiments, a system of ducts and fans is leveraged to create series of measurement chambers. This method will allow for dilution as needed to ensure the ranges of each sensor are adequate, variable flow rates, and exhausting of gasses altered by the measurement process. Air sensing will be of similar importance, and multiple airflow sensors may be required.

In various embodiments, methane sensor may be an optical sensor, calorimetric sensor, pyroelectric sensor, semiconducting metal oxide sensor, and/or electrochemical sensor. Any sensor described herein may be configured to measure and record data at a sampling rate of 1 Hz or higher. For example and without limitation, methane sensor may be an INIR-ME5%. In various embodiments, the oxygen sensor may be a 523-SG-7OX. In various embodiments, the carbon dioxide sensor may be a SCD41-D-R2. In various embodiments, the hydrogen sensor may be a PS1-H2-1000 (TC-1326-AS). In various embodiments, the airflow sensor may be an induction sensor.

In various embodiments, the temperature and humidity sensor may be a SHT40-AD1B-R2. The system may measure ambient temperature from −20 to 60 Degrees Celsius. The system may have a temperature accuracy of +/−0.3 Degree Celsius. The system may have a temperature drift of less than 1 Degree Celsius per year. The system may have a temperature response time of less than 5 seconds for changes to ambient temperature. The system may expose the temperature sensor to ambient air with adequate insulation from the animal. The system may measure ambient temperature changes at 10 second intervals. The system may measure ambient relative humidity levels. The system may measure relative humidity levels between 0 and 99% relative humidity. The system may have a relative humidity measurement accuracy of better than 2%. The system may have a relative humidity drift of better than 1% per year. The system may have a relative humidity response time to changes in ambient relative humidity of 5 seconds or less.

Temperature and humidity sensors are reviewed together as a dual package system is an easy way to reduce design complexity without impacting performance. For this application, the part with the best temperature sensitivity was selected for consideration. SHT40-AD1B-R2 has a temperature accuracy of +/−1 degree Celsius and a relative humidity accuracy of +/−1.8%. The low power consumption and low cost are added benefits. This part works from −40 to 135 Degrees Celsius and up to 100% RH. In various embodiments, Texas Instrument's HDC Line. This line has a number of sensitivities and packages and a part representing temperature and humidity sensing was selected. 595-HDC1080DMBR offer+/−0.2 degree Celsius temperature monitoring and +/−2% RH monitoring.

In various embodiments, any sensor 408 may be configured to record movement of the ruminant or turn on another component of the system. Sensor 408 may be configured such that feeding events can be recorded, and optionally used as a trigger for addition sensing/recording. This serves two purposes 1) it ensure that eructation events will be adequately recorded even after reduction or elimination of methane emissions and 2) it provides a trigger should a low power non-sampling state be needed to meet the life-cycle and power requirements of the system. An initial review suggests that with farm location information (including location and altitude) only a 3-axis accelerometer would be needed.

In various embodiments, sensor 408 may be more than one sensor operating in concert to measure one or more qualities of the ambient environment or gas. For example and without limitation, sensor 408 may be a temperature and humidity sensor as described herein above. In various embodiments, sensor 408 may be electrically coupled to a printed circuit board (PCB) 409. PCB 409 may be the same or similar as any PCB as described herein. PCB 409 may include any electrical component as described herein, such as screens, toggles, switches, buttons or accelerometers. PCB 409 may be electrically coupled to one or more receivers, transmitters or transceivers configured to send and receive electrical signals from a computing device or other electrical component. PCB 409 may include a processor 414 communicatively and electrically coupled thereto. Processor 414 may manage power as described herein, routing electrical energy to the components electrically coupled to the PCB 409, such as sensor 408, fan 407 or the like. In various embodiments, processor 414 may be configured to control data storage from measurements taken by sensor 408 and transmit said data to one or more computing devices remotely located. In various embodiments, the processor 414 may be configured to power on certain components in response to other signals, for example, a motion sensor may indicate t hath the ruminant is moving, the processor 414 may then command the sensor 408 to begin measuring for gas emission, such as methane from the animal.

With continued reference to FIGS. 4A-4E, methane detection device 400 includes an electrical power component 410. Electrical power component 410 may include any electrical power component configured to provide electrical energy to the other components of device to power said components. Electrical power component may be a battery such as a replaceable electrochemical battery cell (i.e., D, C, AA or AAA batteries). Electrical power component may include a rechargeable battery such as a lithium-ion battery. Electrical power component may include a plug port configured to charge said battery by a wall outlet at the amperage and voltage in a residential home. Electrical power component may include a motion-charged battery configured to charge itself based on the motion of said electrical power component, such as along with the movement of a ruminant's head during daily activity (i.e., eating, drinking, walking, turning of the head, flicking of the ears). Methane detection device 400 may include shielding around electronics, sensors, or other sensitive components, the shielding configured to protect said components from electromagnetic interference such as signals from a transceiver, antenna, or emitter. Electrical power component may be configured to last ˜30 days between charges or battery replacement, in some embodiments.

Referring to FIG. 4B-4D, device 400 includes an intake 411. Intake 411 may be formed in the first ring portion 401. The intake 411 may be formed as a plurality of openings disposed through the wall of the first ring portion 401 proximate the filter 404. In various embodiments, when installed on the ruminant, intake 411 may be disposed within the nasal passage of the ruminant. In various embodiments, intake 411 may be disposed proximate the nostrils for the ruminant. In various embodiments, intake 411 may be a single opening disposed within first ring portion 401. In various embodiments, intake 411 may be a series of openings in both the first and second ring portions 401, 402. Device 400 may include an exhaust (not shown). Although not shown in the FIGS. 4A-4B, intake 411 may be disposed on both lateral sides of first ring portion 401 (as can be seen in FIG. 4C) and/or both sides of second ring portion 402.

Referring to FIGS. 4C-4E, device 400 may include an exhaust 412. Exhaust 412 may be disposed proximate or integral to fan 407. Exhaust 412 may be configured to conduct gas out of the housing 403 after measurement. In various embodiments, exhaust 412 may be formed as a plurality of holes in a wall of housing 403. In various embodiments, exhaust 412 may be formed as a single hole in a wall of housing 403. In various embodiments, exhaust 412 may be formed as a slot or series of slots in the wall of housing 403. In various embodiments, exhaust 412 may be formed in one or more of the ring components. In various embodiments, exhaust 412 may be formed in both walls of the housing 403.

Referring specifically now to FIG. 4D, monitoring device 400 may include ambient air intake 413, which can used as a parameter (e.g. baseline value) in the calculation of methane content exhaled from the animal. Ambient air intake 413 may be configured to transmit air from ambient into housing 403. In various embodiments, there may be more than one opening through housing 403 configured to transmit air from ambient into housing 403. In various embodiments, ambient air intake 413 may be mechanically coupled to housing 403. In various embodiments, ambient air intake 413 may be configured to wrap from a first surface of housing 403 to a side surface of housing 403. One of skill in the art would appreciate that when mounted, ambient air intake 413 may be configured to wrap from a bottom surface of the housing 403 to the side of housing 403. In various embodiments, ambient air intake 413 may include an ambient air filter 413a. For example and without limitation, ambient air filter 413a may be formed from 60-65 PPI foam and/or hydrophobic. In various embodiments, any filter as disclosed herein may be utilized as ambient air filter. In various embodiments, ambient air filter 413a may be configured to remove particulate from the ambient air as it travels through ambient air intake 413 into housing 403.

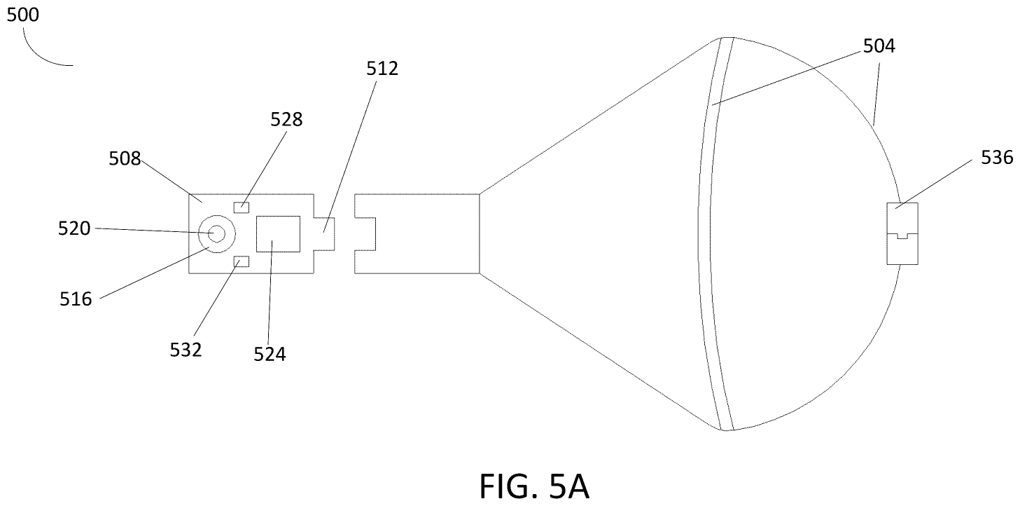

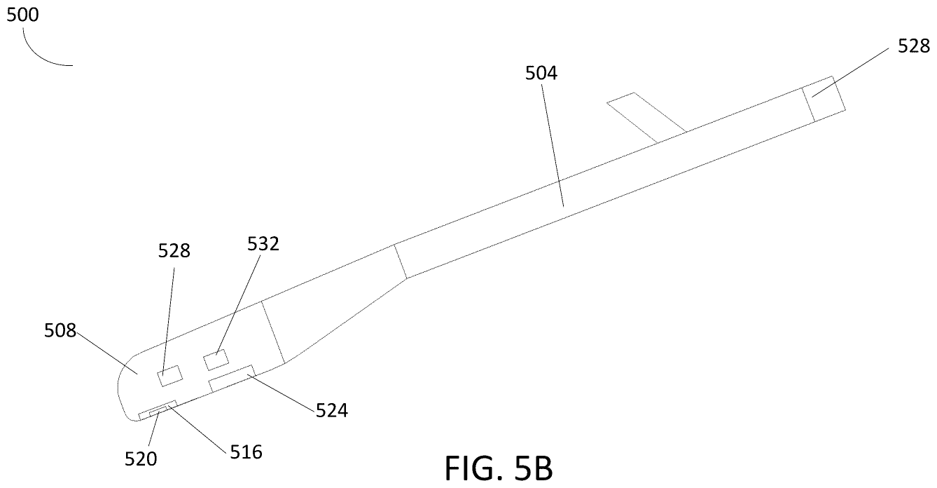

As shown in FIG. 5A, the continuous methane monitoring device includes a harness component 504. Harness component 504 can be configured to adjustably and releaseably fix device 500 to an individual ruminant's head. In some embodiments, the animal is a ruminant as described herein, preferably a cow. In some embodiments the animal is a cow. In some embodiments the animal is a horse. In some embodiments the animal is a donkey. In some embodiments the animal is a mule. In some embodiments the animal is a bull. In some embodiments the animal is a deer. In some embodiments the animal is a chicken. In some embodiments the animal is a goat/sheep/ram. In some embodiments the animal is a pig/hog/boar. In some embodiments the animal is a rooster. In some embodiments the animal is an ox. In some embodiments the animal is a buffalo. The harness component can be configured to attach to any suitable ruminant, preferably a ruminant, such as cattle (e.g., large domesticated ruminant animals, e.g., cows (including dairy cattle), bulls), all domesticated and wild bovines (i.e., those belonged to the family Bovidae; e.g., cows, bulls, bisons, yaks, African buffalos, water buffalos, antelopes), goats, sheep, giraffes, deer, caribou, and gazelles. In preferred embodiments, the ruminant is a cow, a goat, or a sheep. In more preferred embodiments, the ruminant is a cow. One of ordinary skill in the art would appreciate that the continuous methane monitoring device 500 may be configured for use with a plurality of animals/livestock, and this is a non-exhaustive list.

For example and without limitation, harness 504 may include one or more straps extending at least partially around a ruminant's head and secured with a buckle, such as a quick attach/release plastic buckle with pinch sides. Harness 504 may include one more padded straps configured to cushion the abrasion around the ruminant's head, especially around corners and features of the skull, face, horns, neck, ears, eyes, beak, crest, or other features of the animal. Harness 504 may be formed from nylon, cotton, ballistic nylon, leather, Kevlar, wool, plastics, rubbers, a combination thereof, or another material not listed herein. Harness 504 may include an elasticity configured to stretch around the features of the ruminant's head and tighten when in place. Harness 504 may include one or more adjustment features like straps and buckles, double D-rings, pull tabs, holes, slots, hooks and loops, or the like. Harness 504 may include one or more settings, sizes, or shapes configured to secure around the head of an animal, such as a ruminant, or a plurality of successive animals, of the same species or otherwise. Harness 504 may include a plurality of attachment points along its length for additional straps/pads/features configured to better secure around a ruminant's head.

With further reference to FIG. 5A, harness 504 may include a buckle 528. Buckle 528 may be a quick-attach/release buckle configured to be detached using a pinching motion along parallel tabs. Buckle 528 may include a male and female end, the male end configured to pass partially through the female end and change shape in a manner that does not allow movement in the opposite direction. For example the male end may include tabs that are deflected inward by the female end up until a certain point, where the tabs are then elastically returned to their position and disposed in slots of holes in the female end, thereby securing the two together. Buckle 528 may include a hook and loop configuration. In some embodiments, buckle 528 may include Velcro. Buckle 528 may include a belt buckle configuration, wherein one side includes holes at regular intervals and one side includes a pin configured to travel through said hole and hold the first side against a buckle. Buckle 528 may include a pincer configuration wherein a strap is pinched between two metal tongues, thereby securing two sides of the harness together.

With continued reference to FIG. 5A, device 500 includes monitoring component 508. Monitoring component 508 may be one or more housings configured to be releaseably fixable to harness 504. Monitoring component 508 may include a plastic housing with a plurality of cutouts within it, each cutout corresponding to one or more components as described herein. Monitoring component 508 may be formed from one or more plastics, rubbers, metals, composites, or a combination thereof. Monitoring component 508 may be cast, molded, carved, machined, additively manufactured (3D printing), built, assembled, or otherwise formed.

Monitoring component may include a plurality of weight-saving features such as cutouts, carve outs, recesses, or the like. Monitoring component 508 may include one or more ribs assembled into it or manufactured to increase strength. Monitoring component 508 may include a quick attach feature 512. Quick attach feature 512 may allow a user to quickly detach the “smart” component of the device 500 from harness 504, which may still be attached to the cow, and perform a plurality of functions and/or switch the monitoring device 508 with another. Quick attach feature 512 may include a buckle, pull tab, push button, or other elastically deformed component configured to secure with a recess until deflected back and removed. Quick attach feature may be configured for use with one hand, such as a pinching motion. Quick attach feature 512 may be configured to secure into harness 504 with one hand, such as slidably connecting.

With continued reference to FIG. 5A, monitoring component 508 may include intake 516. Intake 516 may include one or more cutouts within monitoring component 508. Intake 516 may be one or a plurality of openings configured to intake a gaseous sample, such as air. In some embodiments, intake 516 is configured to be exposed to the air when device 500 is in use, such that it is not sandwiched between the device and the ruminant's skin. Intake 516 may be disposed facing the nostrils of the ruminant, such as cow's snout, in some embodiments. Intake 516 may include an intake for each of the two nostrils of a ruminant. Intake 516 may be configured to mirror the shape of the ruminant's nostrils and/or mouth. In some embodiments, the monitoring component can include a positioning features (e.g. weights distributed at the bottom/center of the device; contoured outer surface to engage animal anatomy) which act to bias/urge the device into a preferred position on the animal such that the intake 516 is properly aligned with the ruminant's orifice(s) (e.g., nostrils).

Intake 516 may include a vapor barrier to prevent humidity in the air from entering the monitoring component 508. Intake 516 may include a humidity sensor configured to measure the moisture in the exhaled air. Intake 516 may include software and/or hardware configured to manipulate/compensate the measurements to account for environmental factors such as humidity when measuring gaseous compounds. For example and without limitation, intake 516 may include any compensation hardware/software based on the sensor type utilized in said embodiment. Intake 516 may include a plurality of intakes designated for each nostril/mouth. Intake 516 may include a nozzle (e.g., tube or straw) configured to enter the nostrils of the animals to intake exhaled air. Intake 516 may include tubes or straws configured to move in and out of a ruminant's nostrils upon command, upon detection of one or more compounds, or periodically. The intake 516 may include tubes/straws or another form of invasive intakes 516 configured to enter the livestock's nostrils. Intake 516 may extend into monitoring component 508 and include one or more sensors 520 as described herein along said tubes, as is shown in FIGS. 3, 4, 5.

Intake 516 may include one or more turns and be configured to manipulate the gaseous sample taken in. For example the shape of the internal tubing of intake 516 may be used to separate methane from other gases within the exhaled air by gravity or in an active process like an absorber. Intake 516 may include one or more fans configured to assist air in entering the monitoring component 508. The fan used may include a propeller type fan, a bladeless fan, or another type of fluid movement device, like a pump.

With continued reference to FIG. 5A, intake 516 may include one or more absorption agents configured to capture one or more compounds found within the air taken into the system. The absorption compound may be configured to increase a local concentration of captured compounds/elements for downstream processes. In some embodiments, intake 516 may include a flow meter configured to measure the flow of incoming air into intake 516. The flow meter may be communicatively coupled to one or more computing systems, smartphones, electronic devices, or data storage systems on or off board the device 500. The flow meter may be configured to power on or off one or more other components of device 500 based on the measurement of incoming air. For example and without limitation, flow meter may turn on the intake fans when it detects exhaled breath from the ruminant's nostrils, thereby taking in the air.

In some embodiments, monitoring component 508 may include an outlet. The outlet may be one or more ports within monitoring component 508 configured to exhaust processed air. The outlet may include one or more fans configured to pull air through monitoring component 508. The outlet may include one or more absorption agents configured to capture one or more compounds before exhausting air back into the environment. Outlet may include an open hole, opening, cutout or other pass through in monitoring component 508 such that air may pass through the intake 516 and out of the outlet.

Optionally, the monitoring component 508 can include an indicator to confirm (visually, audibly, and/or tactile/haptic feedback) to an operator that the device is properly positioned on the animal with the intake 516 receiving a flow of exhaled methane from the animal. In some embodiments, the device can include an alert/alarm system to notify the operator if/when the device is misplaced, which can be determined based on an absent/interrupted flow at the intake 516.

With continued reference to FIG. 5A, device 500 further includes at least one sensor 520. Sensor 520 may be disposed within at least a portion of intake 516 and in the path of exhaled air being drawing into intake 516. Sensor 520 may be disposed along the wall of a tube within monitoring component 508. Sensor 520 may include a plurality of sensors working in tandem such as a sensor suite to detect methane and quantify the amount of methane present in the sampled air. Sensor 520 may include an infrared sensor, a photoacoustic sensor, ultrasonic sensor, electrochemical sensor, metal-oxide-semiconductor (MOS) sensor, or a combination thereof, among others. One or more other sensors may be used that operate according to semiconductors, oxidation, catalytic reactions, photoionization, infrared, or a combination thereof. For example and without limitation, sensor 520 may include an electrochemical gas sensor. Electrochemical gas sensors may measure the concentration of a target gas, such gaseous methane by oxidizing or reduction the target gas at an electrode and measuring the resulting current. Sensor 520 may be a photoionization detector (PID) configured to measure volatile organic compounds and other gases in concentrations such as parts per billion to parts per million. Sensor 520 may produce instantaneous readings, operate continuously, and are commonly used as detectors for gas chromatography or as hand-held portable instruments.

Further, sensor 520 may be a photoacoustic and/or ultrasonic sensor. This type of sensor may detect the acoustic emission created when a pressured gas expands in a low pressure area through a small orifice, such as a gas entering the intake 516. The photoacoustic sensor may detect the presence of methane or another gaseous compound and the concentration thereof, whether itself or in tandem with another sensor or sensor suite.

Further, sensor 520 may be a holographic gas sensor. Holographic gas sensor may use light reflection to detect changes in a polymer film matrix containing a hologram. Since holograms reflect light at certain wavelengths, a change in their composition can generate a colorful reflection indicating the presence of a gas molecule. However, holographic sensors require illumination sources such as white light or lasers, and an observer or CCD detector. A holographic sensor is a device that may include a hologram embedded in a smart material that detects certain molecules or metabolites. This detection is usually a chemical interaction that is transduced as a change in one of the properties of the holographic reflection (as in the Bragg reflector), either refractive index or spacing between the holographic fringes. The specificity of the sensor can be controlled by adding molecules in the polymer film that selectively interacts with the molecules of interest. A holographic sensor aims to integrate the sensor component, the transducer and the display in one device for fast reading of molecular concentrations based in colorful reflections or wavelengths.

The holographic sensors can be read from a fair distance because the transducer element is light that has been refracted and reflected by the holographic grating embedded in the sensor. Therefore, they can be used in industrial applications where non-contact with the sensor is required. For example, sensor 520 may be configured to be sensitive to methane at about ˜500 ppm (parts per million) and delta (or change over a time period) of +/−5% concentration.

With continued reference to FIG. 5A, device 500 further includes electrical power component 524. Electrical power component 524 is configured to provide electrical energy to the other components of device 500 to power said components. Electrical power component 524 may be a battery such as a replaceable electrochemical battery cell (i.e., D, C, AA or AAA batteries). Electrical power component 524 may include a rechargeable battery such as a lithium ion battery. Electrical power component 524 may include a plug port configured to charge said battery by a wall outlet at the amperage and voltage in a residential home. Electrical power component 524 may include a motion-charged battery configured to charge itself based on the motion of said electrical power component 524, such as along with the movement of a ruminant's head during daily activity (i.e., eating, drinking, walking, turning of the head, flicking of the ears). Monitoring component 508 may include shielding around electronics, sensors, or other sensitive components, the shielding configured to protect said components from electromagnetic interference such as signals from a transceiver, antenna, or emitter. Electrical power component 524 may be configured to last ˜30 days between charges or battery replacement, in some embodiments.

Further, electrical power component 524 may include a photovoltaic cell configured to charge the battery. A solar cell, or photovoltaic cell, is an electronic device that converts the energy of light directly into electricity by the photovoltaic effect, which is a physical and chemical phenomenon. It is a form of photoelectric cell, defined as a device whose electrical characteristics, such as current, voltage, or resistance, vary when exposed to light. Individual solar cell devices are often the electrical building blocks of photovoltaic modules, known colloquially as solar panels. They can be used as a photodetector (for example infrared detectors), detecting light or other electromagnetic radiation near the visible range, or measuring light intensity.

The photovoltaic cell may be disposed on the top most surface of monitoring component 508, the top surface facing the sun when in installed on the livestock animal. The photovoltaic cell may be disposed on the monitoring component 508, on a different part of the livestock (such as on the top of the head, the back, or another area disposed on the animal). The photovoltaic cell, if located off board the monitoring component 508 may be connected via one or more wires to monitoring component 508.

With further reference to FIG. 5A, device 500 includes a data storage component 528. Data storage component 528 may include one or more components located on-board monitoring component 508. Data storage component 528 may include one or more chips, processors, solid state drives (SSD), hard disk drives (HDD), flash memory devices, optical storage devices, or a combination thereof configured to store data. The data stored may include one or more of identified gases and/or concentrations thereof. The data may also include environmental information and time stamps for downstream data processing, such as humidity compensation. Data may be digital data that may be machine-readable on a storage medium, such as data storage component 528. Data may be stored on or in data storage component 528 in raw form, processed, pre-processed, or time stamped such that one or more downstream components or users can visualize the data over time. For example and without limitation, data stored on data storage component 528 may be stored as visual data such as one or more charts. The charts may plot gas detection over time, concentration over time, or some other characteristic of the measured gas or gases. Monitoring component 508 may include a port for insertion of a USB, lightning, thunderbolt, or other electronic storage device external to device 500. Data storage component 528 may be centrally located off board of the device 500 and configured to receive data from one or more monitoring components 508 working in tandem or alone.

With continued reference to FIG. 5A, device 500 includes transceiver 532. Transceiver 532 is configured to transmit data, whether stored data (in data storage component 528) or instantaneously measured data for immediate transmission. In radio communication, a transceiver is an electronic device which is a combination of a radio transmitter and a receiver, hence the name. It can both transmit and receive radio waves using an antenna, for communication purposes. The term is also used for other devices which can both transmit and receive through a communications channel, such as optical transceivers which transmit and receive light in optical fiber systems, and bus transceivers which transmit and receive digital data in computer data buses. Transceiver 532 may be configured to transmit the data, whether stored or instantaneously after measurement, to one or more off-board storage component such as a centralized database for analysis. Transceiver 532 may be configured to transmit data over a plurality of ranges, such as long range communication over satellites, radio or cell towers, over the internet (such as over a WiFi or Ethernet connection), a combination thereof, or the like.

Referring now to FIG. 5B, device 500 is shown in side orthogonal view. One of ordinary skill in the art would appreciate that this is merely a different view of the device as shown in FIG. 5A, and therefore includes at least all of the components as described with reference to FIG. 5A. This side view shows the device including contours that may be complementary to the slope and shape of the head of a ruminant, herein depicted as a cow, for example. One of ordinary skill in the art would appreciate that the device 500 (including harness 504 and monitoring component 508) may be shaped for use with a certain species, or for an individual animal. This side view shows harness 504 including a cross member strap configured to cross over the horn section of the cow's head, whether cow's horns are present or not (such as in cow versus a bull embodiment). The view of FIG. 5B also shows monitoring device 508 installed on harness 504, utilizing the quick attach feature 512 (not shown). According to embodiments described herein above, the photovoltaic cell, if present would be on the top most surface (going into and out of page) as that corresponds to the topmost face of the device 500.

With further reference to FIG. 5B, transceiver 528 and data storage 532 are shown by boxes on seemingly the surface of monitoring component 508, but this does not limit the location of those components. For example, transceiver 528 and data storage 532 may be disposed at the center most portion of the internal space of monitoring component 508 as to protect those components from jostling and damage from day-to-day movement and bumping of the ruminant's head.

With further reference to FIG. 5B, sensor 520 is shown at the exterior-most portion of intake 516, but this is merely for illustration, and the sensor 520 or plurality thereof may be interior to monitoring component 508 as shown in FIGS. 3 and 4. In some embodiments, intake 516 may include a straight tube, as shown in FIG. 3, wherein air is captured and travels through intake 516 past a sensor 520, such as an infrared of photoacoustic sensor, then out of an outlet. This is merely an example of the internal geometry of intake 516 and the traveled path of air exhaled by the livestock and analyzed by the system.

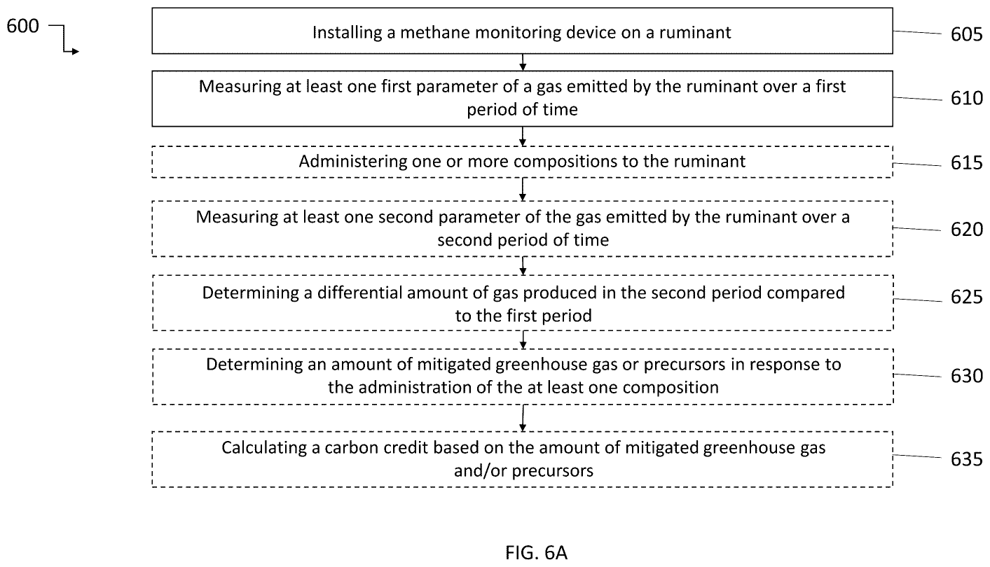

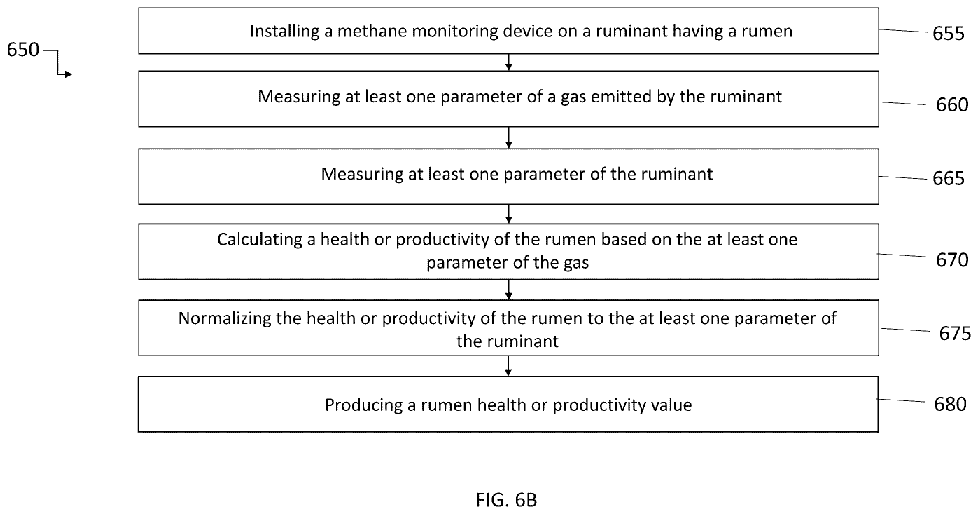

Referring now to FIG. 6A, a method for monitoring methane is shown in flowchart form. Method 600, at step 605 includes installing a methane monitoring device on a ruminant having a rumen. The methane monitoring device may be any as described herein. For example and without limitation, the device may be device 100 and/or device 400. In various embodiments, components of both devices 100 and 400 may be utilized, for example, the ring embodiment of device 400 may be utilized with the neck board 108 of device 100. This disclosure does not seek to limit the methane monitoring device suitable for use with method 600. In various embodiments, installing the device on a ruminant includes strapping the device on the head of the ruminant via straps or harnesses, as described herein. In various embodiments, installing the device on a ruminant includes installing the device within the body of the animal, for example, piercing the septum of the animal with the ring embodiment and hanging the device from the nose of the ruminant. In various embodiments, installing the device on the ruminant may include overlaying one or more components on the back of the animal, the foot or legs of the animal, or any suitable location. In various embodiments, installing the device on the ruminant may include muzzling the animal or installing a device in the mouth or throat of the animal. In various embodiments, the ruminant may be a cow or a sheep or another ruminant as described herein or otherwise.

With continued reference to FIG. 6A, method 600 includes, at step 610, measuring at least one first parameter of a gas emitted from the ruminant over a first period of time. In various embodiments, the gas is a greenhouse gas or a precursor thereof. In various embodiments, the gas is at least a portion methane. In various embodiments, the gas emitted from the ruminant is exhaled or eructed from the ruminant. In various embodiments, the first parameter may be any described herein, for example, composition of matter, detection of a gas, density of the gas (for example in PPM), temperature, humidity among others. In various embodiments, measurements may be taken over the first period of time, the period of time may be between 1 day and 1 year. In various embodiments, the first period of time may be adjusted on a per animal basis or time of year. For example and without limitation, the first period of time may be between 2 weeks and 6 months. In various embodiments, measurements may be taken constantly throughout the first time period or another pattern of measurement, for example, once a day over the time period or for an hour a day during the first time period. In various embodiments, measuring at least one first parameter of a gas emitted from the ruminant may include measuring at a frequency of approximately 0.1-1000 samples per second over the first period.

With continued reference to FIG. 6A, method 600 incudes, at step 615, administering one or more compositions to the ruminant. In various embodiments, administering one or more compositions to the ruminant may include at least of a feed additive, a small molecule inhibitor and/or a vaccine. In various embodiments, administration of the composition may include spraying feed with the composition and allowing the ruminant to eat said feed. In various embodiments, a vaccine may be injected subcutaneously or intravenously to the ruminant. In various embodiments, the composition may be administered orally to the ruminant via a pill, tablet or other solid delivery mechanism ingested alone or in feed. In various embodiments, administering at least one composition to the ruminant includes administering more than one composition, for example a feed additive and a vaccine. In various embodiments, administering at least one composition to the ruminant includes administering more than one composition, for example a feed additive and a small molecule inhibitor. In various embodiments, administering at least one composition to the ruminant includes administering more than one composition, for example a small molecule inhibitor and a vaccine. In various embodiments, the at least one composition may be configured to reduce the amount of gas emitted from the ruminant.

With continued reference to FIG. 6A, method 600 includes, at step 620, measuring at least one second parameter of the gas emitted from the ruminant over a second period of time. In various embodiments, the second period of time is measured after the administration of at least one composition to the ruminant. In various embodiments, the at least one composition may be configured to reduce the amount of gas emitted from the ruminant. In various embodiments, the second period of time may be equal to the first period of time in duration. In various embodiments, the second period of time may be different than the first period of time in duration. In various embodiments, measurements may be taken over the second period of time, the period of time may be between 1 day and 1 year. In various embodiments, the second period of time may be adjusted on a per animal basis or time of year. For example and without limitation, the second period of time may be between 2 weeks and 6 months. In various embodiments, measurements may be taken constantly throughout the second time period or another pattern of measurement, for example, once a day over the time period or for an hour a day during the second time period. In various embodiments, measuring at least one first and second parameter of a gas emitted from the ruminant may include measuring at a frequency of approximately 0.1-1000 samples per second over the first period and second. In various embodiments, measuring the at least one second parameter includes measuring any parameter described herein, for example, an amount of methane emitted from the ruminant over the second period of time. In various embodiments, measuring the amount of methane emitted from the ruminant includes integrating between approximately 10 and 100% of the measurements over the first period or the second period of time.

With continued reference to FIG. 6A, method 600 includes, at step 625, determining a differential amount of gas emitted in the second period compared to the first period. In various embodiments, the total amounts of gas emitted in the first period and the second period may be subtracted from one another to produce a differential amount of gas produced between the first period and the second period. In various embodiments, the administration of the at least one composition may demarcate the first period from the second period, such that the differential amount of gas may be attributed to the composition. For example, a vaccine administered to the ruminant may decrease the amount of gas (including methane) produced by the ruminant. In various embodiments, the differential amount of gas may be displayed to one or more users or stored as data in one or more data stores. In various embodiments, the measurements in the first period and the second period may be normalized or scaled for comparison purposes. For example, and without limitation, the measurements may be taken as a gas emitted per time period, for example per second over the measurement periods.

With continued reference to FIG. 6A, method 600 includes, at step 630, determining an amount of mitigated greenhouse gas or precursors in response to the administration of the at least one composition. In various embodiments, the total amounts of gas emitted in the first period and the second period may be compared to a projection based on animal metrics and environmental data such as temperature, weather and diet. The totals may be subtracted or compared of the projections to produce an amount of mitigated gas. In various embodiments, the administration of the at least one composition may demarcate the first period from the second period, such that the mitigated amount of gas may be attributed to the composition. For example, a vaccine administered to the ruminant may decrease the amount of gas (including methane) produced by the ruminant. In various embodiments, the mitigated amount of gas may be displayed to one or more users or stored as data in one or more data stores. In various embodiments, the measurements in the first period and the second period may be normalized or scaled for comparison purposes. For example, and without limitation, the measurements may be taken as a gas emitted per time period, for example per second over the measurement periods.

With continued reference to FIG. 6A, method 600 includes, at step 635, calculating a carbon credit based on the amount of mitigated greenhouse gas and/or precursors. In various embodiments, calculating carbon credit based on the mitigated gas may include multiplying the amount of mitigated gas by the market rate of at the time of calculation. For example, one carbon credit may be equal to one metric ton of greenhouse gas mitigated. The amount of mitigated gas may be normalized by activity data, emission factor or the like. The amount of mitigated gas may be manipulated or adjusted to carbon dioxide equivalent (CO2e) for calculation of carbon credits. For example, the emission factor of the emitted gas, methane for example, may be multiplied by the global warming potential (GWP) to produce the methane's CO2e. In various embodiments, the calculation of carbon credits may be partially or fully automated, and continuous calculated. For example, the devices installed on the animals may transmit the emitted gas data to one or more computing devices described herein where constant calculations take place to produce or predict carbon credits from the mitigated gases.

Referring now to FIG. 6B, a method 650 for monitoring rumen health is shown in flow chart form. Method 650 includes, at step 655, installing a methane monitoring device on a ruminant having a rumen. Step 655 may be the same or similar to step 605 of method 600. The methane monitoring device may be any as described herein. For example and without limitation, the device may be device 100 and/or device 400. In various embodiments, components of both devices 100 and 400 may be utilized, for example, the ring embodiment of device 400 may be utilized with the neck board 108 of device 100. This disclosure does not seek to limit the methane monitoring device suitable for use with method 600. In various embodiments, installing the device on a ruminant includes strapping the device on the head of the ruminant via straps or harnesses, as described herein. In various embodiments, installing the device on a ruminant includes installing the device within the body of the animal, for example, piercing the septum of the animal with the ring embodiment and hanging the device from the nose of the ruminant. In various embodiments, installing the device on the ruminant may include overlaying one or more components on the back of the animal, the foot or legs of the animal, or any suitable location. In various embodiments, installing the device on the ruminant may include muzzling the animal or installing a device in the mouth or throat of the animal. In various embodiments, the ruminant may be a cow or a sheep or another ruminant as described herein or otherwise.

With continued reference to FIG. 6B, method 650 includes, at step 660, measuring at least one parameter of a gas emitted by a ruminant. The at least one parameter of the gas may be any as described herein. For example and without limitation, the parameter may be amount of gas emitted, density, humidity, composition, frequency of emission of the like. In various embodiments, the gas is a greenhouse gas or a precursor thereof. In various embodiments, the gas is at least a portion methane. In various embodiments, the gas emitted from the ruminant is exhaled or eructed from the ruminant. In various embodiments, the parameter may be any described herein, for example, composition of matter, detection of a gas, density or amount of the gas (for example in PPM), temperature, humidity among others.

With continued reference to FIG. 6B, method 650 includes, at step 665, measuring at least one parameter of the ruminant. In various embodiments, the at least one parameter of the ruminant includes temperature, standing time, laying time, feeding time, quantity eaten, drinking time, quantity drank, behavioral patterns or movement patterns, blood pressure or other vitals measurements including heartbeat, blood sugar or the like. The at least one parameter of the ruminant may include hormonal markers or other chemical measurements. In various embodiments, the at least one parameter may include location of the ruminant within the facility or field, proximity to tother ruminants, mating or other natural behaviors. In various embodiments the at least one parameter may include biometric indicators such as fat content, caloric intake or metabolism related data. In various embodiments, the at least one parameter of the ruminant may include individual identification or genetic information on breeding, lineage or familial relations.

With continued reference to FIG. 6B, method 650 includes, at step 670, calculating a health or productivity of the rumen based on the at least one parameter of the gas. In various embodiments, the parameter(s) of the gas may be analyzed to predict the health and/or productivity of the rumen at a certain time, over a period of time, over the lifespan of the animal, or another time period. In various embodiments, the health and/or productivity of the rumen may be measured against a prediction of the at least one parameter of the gas, such that a comparison can be made between the measured and predicted parameters. For example, a gas emission quantity prediction may be associated with an individual ruminant and compared to the measured gas emission from the device. A productivity and/or health percentage may be assigned to the rumen based on this comparison. The productivity and/or health of the rumen may be measured or assigned in any suitable fashion, for example a number, a percentage, a percentile, a data point or another relative element of data.

With continued reference to FIG. 6B, method 650 includes, at step 675, normalizing the health or productivity of the rumen to the at least one parameter of the ruminant. Normalizing the productivity and/or health of the rumen to the at least one parameter of the ruminant may include associated a health of the rumen with one or more parameters of the ruminant. For example, a health of the rumen may be associated with an individual animal's grazing data. The individual may have a rumen of a certain health based on tracked quantity eaten, eating time, quantity drank and/or drinking time. As the rumen produces gas based on digestion, the rumen health can be monitored relative to the behavior of the ruminant. A health or productivity of the rumen may therefore be associated or scaled to an individual based on habits and/or the biometric data described above. Therefore, the rumen health or productivity of an individual rumen at a distinct time may be adjusted or scaled based on said ruminant data. For example, an overactive rumen of a ruminant may only be overactive a certain amount of time after overeating, the rumen health may then be adjusted near normal if the overeating is an isolated incident. Therefore the normalization of the rumen health to the parameter(s) of the ruminant may accordingly account of ruminant biology and behavior. In various embodiments, the rumen health or productivity may be normalized to a single parameter of the ruminant. In various embodiments, the rumen health and/or productivity may be normalized to a plurality of parameters of the ruminant individually and averaged. In various embodiments, the rumen health and/or productivity may be normalized to a plurality of parameters that are combined into a compound score.

With continued reference to FIG. 6B, method 650 includes, at step 680, producing a rumen health or productivity value. In various embodiments, the rumen health and/or productivity value may be a standalone element of data, not relative to the animal's biology or behavior. Since the rumen health value may be produced from the relative rumen health from step 670, a rumen health value may be general and already account for the individualistic animals. That is to say, that a rumen health value between animals can be assigned and compared. For example, a higher rumen score is associated with a ruminant with a healthier rumen, accounting for the behaviors that differentiate ruminants and the amount of gas produced by the ruminants. Illustratively, a healthier rumen may be scored even if a ruminant produces an abnormally high quantity of gas, due to the normalization of the relative heath score to the parameters of the ruminant, should the ruminant have overeaten, overdrank or the like. The rumen health value may be transmitted to one or more computing systems or users. The rumen health value may be stored in one or more data stores. In various embodiments, the rumen health value may be stored and transmitted simultaneously to one or more users or computing devices. In various embodiments, the rumen health value may trigger one or more actions by communicatively coupled devices or alert the user to adjust handling of the ruminant, for example, by reducing or increasing feed/water times.

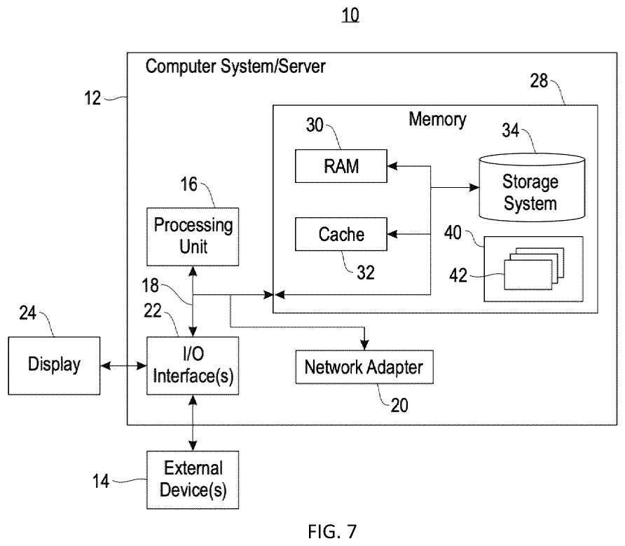

As shown in FIG. 7, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, may be signals, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C4 or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein may include an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which may include one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A methane monitoring device, the device comprising:
a harness component, the harness component configured to adjustably and releasably couple to a ruminant;
a monitoring component releasably coupled to the harness component, the monitoring component including:
a nostril cover, the nostril cover having a top wall, a first sidewall, a second sidewall and a distal wall, wherein the nostril cover is configured for placement proximate the ruminant's nostrils;
at least one sensor configured to detect methane concentration; and
a data storage component in electrical communication with the sensor, the data storage component configured to store data.

2. The methane monitoring device of claim 1, wherein the monitoring component comprises an oxygen sensor and/or a carbon dioxide sensor.

3. The methane monitoring device of claim 1, wherein the monitoring component comprises a pressure sensor.

4. The methane monitoring device of claim 1, wherein the monitoring component comprises a relative humidity sensor and a temperature sensor.

5. The methane monitoring device of claim 1, wherein the monitoring component comprises a transceiver in electrical communication with the data storage component and configured to transmit the data.

6. The methane monitoring device of claim 1, wherein the monitoring component comprises an accelerometer.

7. The methane monitoring device of claim 1, further comprising a neck board, the neck board releasably coupled to the harness component.

8. The methane monitoring device of claim 6, wherein the neck board comprises an ambient air intake and at least one of an oxygen sensor, a carbon dioxide sensor, a pressure sensor, a relative humidity sensor, a temperature sensor, or an accelerometer.

9. The methane monitoring device of claim 8, wherein the neck board comprises an electrical power component electrically coupled to a charge management controller.

10. The methane monitoring device of claim 1, wherein the monitoring component comprises an intake duct, the intake duct coupled to a fan, the fan configured to force gas through the intake duct, the intake duct in fluid communication with an airflow sensor.

11. The methane monitoring device of claim 1, wherein the monitoring component comprises a filter.

12. A methane monitoring device, the device comprises:
a ring component having an inner diameter and an outer diameter, defining a radial thickness therebetween, the ring component including:
a first ring portion having an arcuate shape and a second ring portion having an arcuate shape, each of the first and the second rings portions having a first and a second ends, wherein
the second ring portion is configured to hingedly rotate relative to the first ring portion at the first end, and releaseably couple to the first ring portion at the second end;
a housing coupled to an inner perimeter of the first ring portion, the housing having a generally planar first wall and second wall, defining a cavity disposed therebetween;
at least one sensor disposed within the housing, the at least one sensor configured to detect a concentration of methane.

13. The methane monitoring device of claim 12, wherein the first ring portion has an arcuate hollow portion therein, and a piercing component at the second end thereof.

14. The methane monitoring device of claim 13, wherein at least one filter is disposed within the arcuate hollow portion.

15. The methane monitoring device of claim 12, wherein the housing comprises at least a second sensor disposed therein.

16. The methane monitoring device of claim 12, wherein the housing comprises a fan configured to displace a gas within at least a portion of the ring component or the housing component.

17. The methane monitoring device of claim 15, wherein at least one sensor comprises an oxygen sensor or a carbon dioxide sensor.

18. The methane monitoring device of claim 15, wherein at least one sensor comprises a pressure sensor, a relative humidity sensor, a temperature sensor or an accelerometer.

19. A method for monitoring a gas, the method comprising:
installing a gas monitoring device on a ruminant; and
measuring at least one first parameter of a gas emitted from the ruminant over a first period of time;
administering one or more compositions to the ruminant;
measuring at least one second parameter of the gas emitted from the ruminant over a second period of time;
determining a differential amount of greenhouse gas produced in the second period compared to the first period;
determining an amount of mitigated greenhouse gas and/or precursors thereof in response to the administration of the at least one composition to the ruminant between the first period and the second period;
calculating a carbon credit based on the amount of mitigated greenhouse gas and/or precursors thereof.

20. The method of claim 19, wherein the gas is a greenhouse gas or precursor thereof.

21. The method of claim 19, wherein the ruminant is a cow or a sheep.

22. The method of claim 20, wherein the gas is exhaled or eructed methane from the ruminant.

23. The method of claim 19, wherein the one or more compositions comprises at least one of a feed additive, a small molecule inhibitor, or a vaccine.

24. The method of claim 19, wherein the first period and the second period of time are each between approximately one day and one year.

25. The method of claim 19, wherein the at least one first parameter and second parameter are a quantity of methane emitted from the ruminant.

26. The method of claim 19, wherein measuring the amount of methane emitted from the ruminant comprises integrating between approximately 10-100% of the measurements over the first period or the second period.

27. The method of claim 19, wherein measuring at least one first parameter or the at least one second parameter of a gas emitted from the ruminant comprises measuring at a frequency of approximately 0.1-1000 samples per second over the first period or the second period.

28. A method for monitoring the health or productivity of a rumen, the method comprising:
installing a gas monitoring device on a ruminant having a rumen;
measuring at least one parameter of a gas emitted from the ruminant;
measuring at least one parameter of the ruminant;
calculating a health or productivity of the rumen based on the at least one parameter of the gas;
normalizing the health or productivity of the rumen to the at least one parameter of the ruminant; and
producing a rumen health or productivity value.

29. The method of claim 28, wherein the gas is methane that has been exhaled or eructed by the ruminant.

30. The method of claim 28, wherein the at least one parameter of the ruminant is a temperature, a standing time, a laying time, a feeding time, a quantity eaten, a drinking time, or a quantity drank.
